(19) United States
(12) Patent Application Publication
(10) Pub. No.: US 2025/0372206 A1
(43) Pub. Date: Dec. 4, 2025

(54) Methods, Devices, Computer Readable Storage Media, and Electronic Devices for Obtaining Microbial Species Identity and Related Information by Sequencing

(71) Applicant: Inquire Life Diagnostics, Inc., Xi'an (CN)

(72) Inventors: Shan Jiang, Xi'an (CN); Yumei Yang, Xi'an (CN); Baibing Pang, Xi'an (CN); Christopher Lei Johnson, Xi'an (CN)

(21) Appl. No.: 18/019,995

(22) PCT Filed: Aug. 31, 2021
(86) PCT No.: PCT/CN2021/115705

§ 371 Date: Feb. 6, 2023

(30) Foreign Application Priority Data
Aug. 7, 2020 (CN) 202010790095.7

(51) Int. Cl. G16B 30/10; G16B 40/30

(52) U.S. Cl. CPC G16B 30/10; G16B 40/30

(57) ABSTRACT

This invention relates to the area of microorganism identification, specifically involving a method of obtaining microorganism identities and related information by sequencing. The method includes: i) obtaining sequencing data, said sequencing data are obtained by amplification of microbial characteristic sequences using primers followed by sequencing the amplification products using next-generation sequencing technology; ii) comparing said sequencing data with characteristic sequence database to identify microbial composition in said samples tested; wherein perform clustering on said characteristic sequence database in advance based on the sequence similarity among reference sequences containing said characteristic sequences, obtain one or more tiers of clusters, there is at least one child seed in each cluster, and there are several seeds as reference sequences in the bottom tier cluster.

### FIELD OF THE INVENTION

The present invention relates to the field of microorganism identification, in particular, involving methods, devices, computer readable storage media and electronic devices to obtain identification and relevant information of microbial species through sequencing method.

### BACKGROUND OF THE INVENTION

Microorganisms are classified into eight major groups including bacteria, viruses, fungi, actinomycetes, rickettsia, mycoplasma, chlamydia and spirochete. Next generation sequencing (NGS) and metagenomic NGS (mNGS) technology is an effective method to identify microorganism species in samples.

NGS has been used to identify microorganisms in two major ways:

One is to use metagenomic strategy, by which all nucleic acid sequences isolated from the samples are detected and the presence of organisms in the sample are identified by comparing the detected sequences with the microbial genomic sequence database.

The other is targeted sequencing strategy. Certain characteristic sequences in the sample are specifically captured or enriched, and then sequenced. The sequences obtained are compared with the corresponding microbial characteristic sequence database so as to identify the microorganisms in the samples. The types of prokaryotic rRNA include: 23S, 16S, and 5S rRNA. The genes encoding 16S rRNAs are evolutionarily highly conserved and suitable in length for analysis (about 1540 bp). The sequence diversity correlates well with the evolutionary distance between the two species. Thus 16S rRNA gene has become the standard molecular marker used in bacterial identification. In addition, 16S rRNA gene is not only suitable for the classification of bacteria, but also for the classification of mycoplasma, chlamydia, rickettsia, spirochete and other prokaryotes. It is so far the most widely accepted characteristic sequence for the classification of prokaryotes and has the most complete database. The sequence of 16S rRNA gene contains 9 or 10 highly variable regions and 10 or 11 evolutionarily conserved regions. Conserved sequences reflect the phylogenetic relationships among the biological species, and variable sequences reflect the differences among the biological species. The targeted NGS sequencing strategy is aimed at the highly variable sequences of 16S rRNA genes. PCR-amplified sequences of 100 to several hundred bp are used for NGS sequencing and the sequence information obtained is compared with the 16S rRNA gene sequence database to identify the presence of microorganisms in the sample.

However, when metagenomic next generation sequencing (mNGS) technique is applied to microbial identification, especially in the identification of clinical pathogenic microorganisms, total nucleic acids of samples are sequenced through indiscriminately. Due to the presence of a large number of non-microbial host nucleic acids such as those from human cells in the sample, and that the amount of nucleic acids in a human cell is about 1,000-100,000 times as large as that of a microbial cell, and that only about 1% of genomic sequences are species-specific in microorganisms, in addition, that pathogenic microorganisms constitute a very small fraction in clinical samples as compared to host cells, only 1/1,000,000-100,000,000 of the nucleic acids in the tested samples come from pathogenic microorganisms. As a result, most sequencing data are not relevant for the purpose of microbial identification and are invalid data. The waste of sequencing data, on one hand, leads to the high cost of the test, and on the other hand, reduces the sensitivity and reliability of the test due to insufficient valid data.

NGS technology based on 16S/18S/ITS amplicon has limited read length. Depending on the type of sequencing platforms, the length of sequencing reads ranges between 50-400 bps. However, the length of the 16S rRNA gene is about 1500 bps. In order to obtain the full-length sequence information of the gene, the nucleic acid of the gene must be fragmented into shorter pieces suitable for NGS sequencing. After completing the sequencing, the full-length sequence of the 16S rRNA gene can be assembled by aligning the short fragments according to the overlapping end sequences between different fragments. However, ribosomal gene sequences are highly conserved in evolution. Sequences of species that are evolutionarily close (for example, species within the same genera) are highly similar. Therefore, when a clinical sample contains more than one species, it is likely to have difficulties to assemble full-length 16S rRNA gene sequences from short fragments correctly for each species without creating chimeric sequences due to a high level of sequence similarity among short fragments belonging to different species.

To avoid the above difficulties, the currently popular amplicon NGS technology amplifies the variable regions of 16S rRNA gene followed by NGS sequencing of amplicons. Since the sequences of nine or ten variable regions of 16S rRNA gene reflect the differences among different species, NGS sequencing of one or a few variable regions followed by comparison of sequence obtained with variable region sequence database enable identification of some microorganisms at the “species” level.

However, the nucleotide sequence diversity carried by a single or several variable regions is not sufficient to distinguish all prokaryotic organisms. Johnsons, J. S. et al. (2019) reported that only the full-length nucleotide sequence of the 16S rRNA gene contains enough information to distinguish all prokaryotic organisms at the “species” level. Therefore, the current 16S/18S/ITS amplicon based NGS technology is not capable to identify microorganisms in clinical samples at the “species” level.

To summarize, each of the aforementioned technologies has its own limitation when applied to clinical microbial identification.

### SUMMARY OF THE INVENTION

The present invention relates to a method for obtaining microbial species identities and related information by sequencing in a sample, including:
i) obtaining sequencing data, which is acquired by targeted enrichment/amplification of microbial characteristic nucleic acid sequences followed by sequencing the enrichment/amplification products by an NGS technology;
ii) comparing and analyzing said sequencing data with a characteristic sequence database containing reference sequences to identify the composition of microorganisms in the tested sample;
wherein the reference sequences in said characteristic sequence database are organized in advance by clustering analysis based on sequence similarity to obtain a hierarchical structure comprising one or more tiers of sequence clusters, wherein each cluster has at least one seed sequence (the most representative reference sequence for all reference sequences in the child clusters of this cluster) and each cluster at the bottom tier of the structure has one or more member reference sequences;
wherein said comparing and analyzing includes:
   a) remove low-quality reads and those containing sequencing adaptor sequences from the sequencing data;
   b) align the sequencing reads with the seed sequences of said characteristic sequence database. After removing duplicate reads generated by PCR amplification, perform statistical test for each seed sequence on independence between the events “there are reads aligned with reference sequences” and the seed sequences. All seed sequences of statistical significance comprise a set of primary screened seed sequences;
   c) The sequencing reads are aligned with each of the primary screened seed sequences obtained in b), wherein the primary screened seed sequences do not compete with each other for reads. Determine the read coverage and calculate the metrics characterizing coverage for each seed sequence. Select seed sequences based on their coverage metrics to form a set of secondary screened seed sequences;
   d) Align sequencing reads with the secondary screened seed sequences obtained in c), wherein seed sequences compete against each other for sequencing reads in the process. The read coverage metrics are calculated for each seed sequence, and the seed sequences that do not meet the requirements for read coverage metrics are removed iteratively. Seed sequences that remain after the alignment process are the tertiary screened seed sequences;
   e) Pool together all tertiary screened seed sequences and perform sequence alignment between reference sequences represented by the tertiary screened seed sequences and sequencing reads. Obtain reference sequences that meet the requirements after an iterative screening process, wherein the threshold used in the screening process is more stringent than that used in step d);
   f) Based on the reference sequences and their aligned sequencing reads obtained in step e), Calculate the total read count and the relative abundance in reads at the species level. The total read count for each species can be obtained by summing up the number of aligned reads from all reference sequences belonging to this species. And the relative abundance of each species can be calculated by dividing the total read count for this species by the sum of total read count for each species identified in this sample.

This invention also relates to a device for obtaining microbial species identity and related information by sequencing in a sample, the device comprises:
i) Sequencing data acquisition module, which is used to obtain said sequencing data which are acquired by targeted enrichment of microbial characteristic nucleic acid sequences followed by NGS sequencing the enriched products;
ii) Characteristic sequence database construction module, which performs clustering analysis on said microbial characteristic sequences to construct characteristic sequence database which contains a tree structure comprising one or more tiers of clusters, each of which has at least one seed sequence and the bottom-tier cluster has one or more reference sequences; and
iii) Comparative analysis module, which performs sequence alignment between said sequencing data and said characteristic sequence database to identify the microbial composition in the tested sample. Said comparative analysis is defined as the comparative analysis in the method described above.

In one aspect, this invention also relates to a computer readable storage medium, which herein is used for storing computer instructions, programs, code sets or instruction sets which, when runs on a computer, causes the computer to perform step ii) of the method described above.

In another aspect, this invention also relates to an electronic device, including:
i) One or more processors; and
ii) Storage devices that store one or more programs, wherein when said one or more programs are executed by said one or more processors, said one or more processors implement step ii) of the method described above.

This invention also relates to the application of the method, or device, or computer readable storage medium, or the electronic device described above in identification of microorganisms.

Despite long-term efforts, current technologies are still unable to solve satisfactorily the problem of microbial species identification based on sequence information of evolutionarily highly conserved long sequences such as 16S rRNA gene sequence using short-read NGS technology. This invention provides an effective solution for the problem. Tests on laboratory and clinical samples confirm that this invention distinguishes, with accuracy, highly similar long sequences such as those of 16S rRNA gene and so on. This invention overcomes the difficulty that the application of targeted sequencing is limited to tests of short sequences only in the past, and achieves microbial identification at species level or higher resolution based on short-read sequencing.

This invention is able to correctly identify microbial species present in the tested sample and measure the relative proportion that each species occupies with higher accuracy and sensitivity than existing technologies. For example, in the testing of bacteria, the detection limit for a single species can be as low as 10 CFU. This invention can correctly detect the presence of all microorganisms in samples of mixed population of multiple (such as five or more) species even if the concentration difference between any of two species reaches 16 times or greater.

In the tests of clinical samples, the average amount of sequencing data for all samples in example 3 to 9 of this disclosure is 55,663 reads, which is far below the amount of data required (10,000,000-100,000,000 reads) by the current mNGS technology. Over 90% of the data are effectively used in microbial identification. In contrast, only 0.00001-0.01% of the sequencing data is valid in microbial identification using mNGS technology according to research publications. Thus, compared to mNGS, this invention demonstrates high data efficiency. The cost of tests conducted with this invention is much lower than that of the current technology, mNGS.

Relatively high sequencing depth can ensure the accuracy of detection. In the test conducted by this invention, the coverage rate of the target sequence is nearly 100% and only microbial species identification with sequencing depth of 20× or more are accepted. However, in current publications, the coverage requirements for mNGS in microbial detection can be as low as 10%, implicating a lower than 1× average sequencing depth. Therefore, the sensitivity and specificity of this invention for microbial detection are higher than the existing technologies.

Results of tests using cultural and clinical samples proved that this invention provides satisfactory test sensitivity and specificity while ensuring low cost of tests. Thus this invention provides higher test sensitivity and accuracy while preserves the technical advantages of mNGS such as broad target range and resistance to the impact of many irrelevant influences like prior exposure to antibiotics.

### BRIEF DESCRIPTIONS OF THE DRAWINGS

Brief descriptions of drawings for illustrating certain specific embodiments or existing techniques are provided below. Obviously, the drawings referred in the following descriptions are some embodiments of the present invention. For persons of ordinary skill in the art, other drawings can also be obtained based on drawings herein without creative efforts.

FIG. 1 is a schematic diagram that illustrates, in an example of this invention, the process wherein microorganisms are detected by nucleic acid sequencing of prokaryotic 16S rRNA gene.

FIG. 2 is a schematic diagram that illustrates, in an example of this invention, the main operate flow that based on the construction of a 16S rDNA reference sequence database.

FIG. 3 is a schematic diagram that illustrates, in an example of this invention, the construction of a database consisting of two tiers of clustering hierarchy.

FIG. 4 is a schematic diagram that illustrates, in an example of this invention, the hierarchical structure of seed clusters in a reference sequence database.

FIG. 5 is a schematic diagram that illustrates, in an example of this invention, the algorithmic work flow for microorganism species identification using sequencing data.

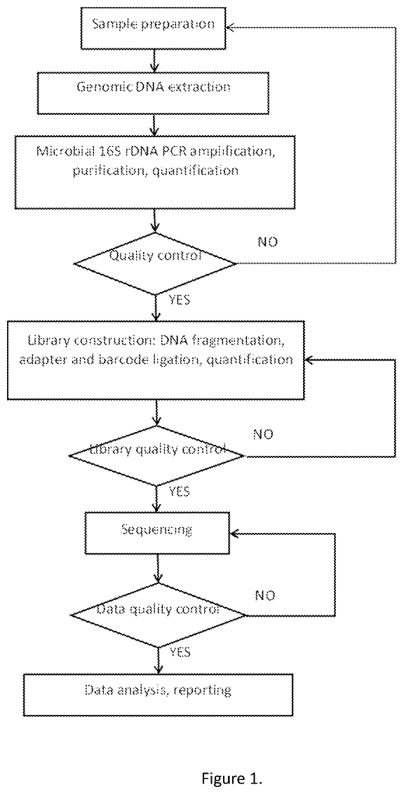

### DETAILED DESCRIPTION OF THE INVENTION

Provided herein are detailed references for embodying this invention, of which one or more examples are described in the following. Each example provided is an explanation rather than a limitation of the invention. In fact, it is obvious to a person of ordinary skill in the field that many modifications and variations may be made to this invention without departing from the scope or spirit of this invention. For example, a feature illustrated or described as part of an embodiment can be used in another embodiment to produce a further embodiment.

The present invention is therefore intended to cover said modifications and variations falling within the scope of the attached claims and its equivalents. Other objects, features and aspects of this invention are disclosed in or are evident from the following detailed description. A person of ordinary skill in this field should understand that this discussion is only a description of exemplary embodiments and is not intended to limit broader aspects of this invention.

This invention relates to a method for obtaining microbial species identity and related information by sequencing, including:
i) Obtaining sequencing data, which is acquired by targeted enrichment/amplification of microbial characteristic nucleic acid sequences followed by sequencing the enrichment/amplification products by an NGS technology;
ii) Said sequencing data are compared with the characteristic sequence database to identify the composition of microorganisms in the tested sample;
The reference sequences in said characteristic sequence database is organized in advance by clustering analysis based on sequence similarity to obtain a hierarchical structure comprising one or more tiers of sequence clusters, wherein each cluster has at least one seed sequence (the most representative reference sequence for all reference sequences in the child clusters of this cluster) and each cluster at the bottom tier of the structure has one or more reference sequences;
Said microorganisms include bacteria, archaea, fungi, mycoplasma, chlamydia, rickettsia, spirochetes, and viruses, wherein the characteristic nucleic acid sequence of RNA viruses can be obtained by reverse transcription of their RNA genomes to generate cDNA.

### Terms and Abbreviations

As used herein, the following abbreviations refer to and the definitions of terms are provided:

NGS=Next-Generation Sequencing.

mNGS=metagenomics Next-Generation Sequencing.

ITS: Internal Transcribed Spacer, which is the nucleic acid sequences located in between sequences of large and small subunit rRNA genes in the transcribed region of the polycistronic rRNA precursor.

Reads: Sequencing reads, refer to individual pieces of sequences produced by NGS.

Cor=Pearson's correlation coefficient.

NRMSE=Normalized root mean square error.

CV=Coefficient of variation.

Fastq: A four-line text file format for storing nucleic acid sequences and their sequencing quality values.

Adapter: Adapter sequence used in sequencing.

Seed or Seed sequence: a reference sequence representative of all reference sequences in a cluster generated by clustering analysis.

Bowtie2: a software that aligns short sequences to large genomes.

Mean depth: averaged sequencing depth.

Gap: a blank or break point in a reference sequence, where no sequencing reads is aligned to.

End gap: a blank at the end of a reference sequence where no sequencing reads is aligned to.

Middle gap: a blank or break point in the middle of a reference sequence where no sequencing reads is aligned to.

EM=Expectation Maximization.

Overlap graphs: a graph representing the sequence overlapping relationship between multiple nucleic acid sequences.

Paired-end reads: sequencing reads generated by forward and backward sequencing of the template nucleic acid fragment.

De novo assembly: a method of assembling sequencing reads from scratch into a longer sequence using short, unorganized reads.

Reference sequence: In the present invention, unless otherwise specified, a reference sequence is a characteristic sequence that can represent a microorganism species, which generally is evolutionarily conserved. The reference sequences commonly include 16S rRNA gene, 18S rRNA gene, ITS nucleic acid sequence, RNA-template RNA polymerase gene (RdRp) of RNA viruses, viral capsid protein coding gene, pol gene of retrovirus and so on, or full-length sequences of one or more other nucleic acid sequences that can characterize microbial species.

### Samples, Preprocessing and Sequencing Library Construction

In the present invention, the subjects of tests may come from a living organism (microbial host) or from an environmental sample containing microorganisms. In some embodiments, the sample to be tested is a sample from a microbial host or from an environment containing microorganisms: said samples from a microbial host include preferably but are not limited to: at least one among faces, intestinal contents, skin, tissue, sputum, blood, saliva, dental plaque, urine, vaginal secretions, bile, bronchoalveolar lavage fluid, cerebrospinal fluid, pleural effusion, ascites, pelvic effusion, pus, and rumen; In some embodiments, samples herein from environments containing microorganisms include preferably: at least one among internal and external surfaces of objects, domestic water, medical water, industrial water, food, beverage, fertilizer, sewer, volcanic ash, permafrost, silt, soil, compost, polluted fish farming river water, aquaculture water bodies and air.

In some embodiments, said hosts are animals; further optionally include human beings and all livestock (such as domestic animals and pets) and wild animals and birds, which include but are not limited to cattle, horses, dairy cattle, pigs, sheep, goats, rats, mice, dogs, cats, rabbits, camels, donkeys, deer, mink, chickens, ducks, geese, turkeys, fighting cocks, etc.

In the example of microbial tests based on prokaryotic 16S rRNA gene sequencing, the testing process is shown in Figure I and detailed as follows:

Sample preparation: Depending on the types and origins of samples, preprocessing may be needed for nucleic acid extraction. Preprocessing methods include, but are not limited to, using sterile water, ddH2O (double distilled water), sterile saline, sterile PBS (phosphate buffer salt solution) and other liquids to wash the sample; using filtration, centrifugation and other methods to concentrate the sample; using gradient centrifugation and other methods to separate some components in the sample; or using some kits that meet the experimental requirements to separate some components in the sample; or remove or enrich the nucleic acid in certain parts of the sample.

Nucleic acid extraction: use nucleic acid extraction kits to extract all nucleic acids of the sample after sample preparation. The nucleic acid extraction kits used is not limited to a certain manufacturer nor is it limited to a certain method, as long as nucleic acids of quality required by the experimental needs can be achieved by the kits. The extracted nucleic acids include DNA, RNA, or both. Prior to this step, a certain amount of nucleic acids of known sequence that satisfy the following conditions can be added to the sample: 1) they can be amplified in the reaction system prepared in the next step; 2) Primers added in the next step or primers prepared separately can anneal to them; 3) The sequences are known; 4) The sequences used will not interfere with the analysis of any target sequences that may exist in the sample; 5) The nucleic acid sequences can exist alone or with carriers such as plasmids, viruses, and cells; 6) The added nucleic acid sequence can be obtained by the operation in this step and is present in the final extracted nucleic acids. In this technical scheme, the addition of nucleic acids of known sequences allows better judgement on existence of contamination introduced into test results by sampling, experimental and/or other processes. However it is not required for this technical scheme. Missing the addition of said nucleic acids of known sequences does not affect the integrity of this technical scheme, nor does the presence constitute an innovation to this technical scheme. The application of adding nucleic acids of known sequences is also not restricted to this step.

Targeted enrichment of specific nucleic acid sequences: certain methods can be used to enrich nucleic acid sequences that can provide information on microbial classification, elevate the proportion of said nucleic acid sequences in the total nucleic acid sequences derived from the sample, and purify and quantify the enriched products. Enrichment methods include but are not limited to PCR amplification, hybridization capturing and other methods thereof. The purification of enrichment products includes but is not limited to affinity column purification, magnetic beads purification and other methods. The purpose of the purification is to remove residual enzymes, primers, probes, salts, metal ions and other components in the sample during the enrichment process and obtain pure nucleic acids of longer fragments (greater than 20 bp). Quantification is to determine the concentration of nucleic acids in the sample, allowing calculation of the quantity of nucleic acids in the sample based on the sample volume. Quantification methods include ultraviolet spectrophotometry, dye binding assay and so on. The enriched target sequences are sequences commonly used for microbial species classification. For prokaryotes, they could be the ribosomal 16s rRNA gene sequences; for eukaryotes, they could be the ribosomal 18s rRNA gene sequences or ITS sequences; for viruses, they could be the nucleic acid sequences that are both evolutionarily conserved and species specific in their genomes, such as Pol (RNA-dependent RNA polymerase) and N (Nucleocapsid) genes in the coronavirus genome. Such sequences are usually long and may contain highly similar portions among different species, whose species origin cannot be differentiated correctly by existing short-read NGS sequencing and data analysis methods. For RNA obtained from the sample, reverse transcription may be needed to reverse transcribe RNA to cDNA before targeted enrichment of specific sequences. In this step, a certain amount of nucleic acids of known sequences (as a positive control) can also be added when preparing the reaction system. Said nucleic acids of known sequences satisfy the following conditions:
1) They can be amplified in the reaction system prepared in this step;
2) They can be annealed to by the existing primers added in this step or the primers prepared separately;
3) They are completely known;
4) They do not interfere with the analysis of any target sequences that may exist in the sample;
5) They can exist alone or with carriers such as plasmids, viruses, and cells.

In some embodiments, nucleic acid enrichment is followed by one or more steps of quality control procedures, the purpose of which is to test the efficiency of enrichment of the target nucleic acid sequences. Methods of quality control include tests of quantity, purity, and fragment length thereof of enriched nucleic acid sequences. Efficient sample enrichment leads to more efficient sequencing, which usually means microbes are more concentrated in the sample. Judging from the enrichment efficiency, the content level of microorganisms in the sample can be predicted. For samples whose enrichment quality does not meet the expectation, the enrichment operation may be re-performed, that is not always necessary, or subsequent experiments follow without repeating the enrichment operation.

Sequencing library construction: The purpose is to convert the enriched nucleic acids into short nucleic acid fragments that can be tested by the NGS platform. The main step is to break the long nucleic acid sequences into fragments of length that can be read by the NGS platform, at the same time, sequencing primers are added to both ends of the fragments so that sequencing machines are able to measure the nucleic acid sequences. Barcode/index in the sequencing primers enables identification of sample sources. Before starting this step, a certain amount of nucleic acids of known sequences can be added to the nucleic acids obtained in the previous step, which satisfies the following conditions:
1) all sequences are known;
2) the sequences used do not interfere with the analysis of any target sequences that may be present in the sample; and
3) nucleic acid sequences can exist alone or with carriers such as plasmids, viruses, and cells.

In the present invention, the addition of nucleic acids of known sequences allows better judgement on existence of contamination introduced into test results by sampling, experimental and/or other processes. However, it is not required for this invention. Missing the addition of said nucleic acids of known sequences does not affect the integrity of this technical scheme, nor does the presence constitute an innovation to this technical scheme. The application of adding nucleic acids of known sequences is also not restricted to this step.

The specific experiments of sequencing library construction include the following steps:
1) Fragmentation of nucleic acids. Long fragments of nucleic acid sequences are broken into shorter fragments. Since the extracted or enriched nucleic acid sequences are still very long, which greatly exceeds the reading capacity of NGS instruments, it is necessary to break the long fragments into short fragments that can be read by NGS instruments and then perform parallel sequencing to obtain sequence information of all the nucleic acids. Methods of fragmentation include, but are not limited to, physical methods (such as the use of ultrasound equipment to disrupt nucleic acids), biological methods (such as the use of nuclease, transposase, and other methods to cut nucleic acid sequences), etc.
2) End repair. In the whole process of library construction, nucleic acids subject to the experimental manipulations are double-stranded DNA (dsDNA). The two ends of short dsDNA generated by fragmentation of long dsDNA are usually not blunt instead have protruding bases on either one of the two strands forming sticky ends. Depending on the sequencing platforms used, it is necessary to repair the broken needs in different ways. For example, if Thermo Fisher's Ion torrent sequencing platform is used, the broken ends need to be repaired to form perfect blunt ends; and in the case of Illumina sequencing platform, the ends should be repaired in a way such that one of the strands has an additional adenine nucleotide (A).
3) Size selection of fragments. Magnetic beads are used to screen nucleic acid fragments in the sample. Only fragments of appropriate sizes are selected while those too long or too short are discarded. The selection size of nucleic acid fragments vary with the sequencing platform, sequencing reagent and sequencing conditions used.
4) Ligation of sequencing adaptors. Sequencing adaptors are two pieces of dsDNA with specific sequences. In a sequencing device, the sequencing reaction starts from where these specific sequences are. Sequencing primers may or may not contain barcode/index sequences. Barcode/Index sequences are used to distinguish sequencing reads generated from different sample sources within the same sequencing experiment. Using enzymatic tools such as T4 ligase, two sequencing primers were ligated separately to each end of a short fragment of dsDNA that were already subject to end repair. Only dsDNA fragments with one sequencing primer attached to each end can be sequenced.
5) Library enrichment. All nucleic acid sequences in a tested sample with correctly ligated sequencing primers are called a sequencing library. Library enrichment is the process that, by a certain method usually PCR, amplifies the number of nucleic acid sequences with correctly ligated sequencing primers to increase their copy number and facilitate subsequent work. The enrichment step is not always necessary in the experimental process.
6) Purification operation follows each reaction in the course of library construction in order to separate nucleic acids from reaction reagents and obtain pure nucleic acids for the next reaction. With adjustments in reagents and reaction conditions, not every purification operation is necessary. Said adjustment is not beyond the scope of this technical scheme, and the reagents and methods used for purification do not constitute limitations of this technical scheme.

In some embodiments, quality control steps are also included after the construction of sequencing libraries, which aim to test whether the constructed sequencing libraries meet the sequencing requirements or not. The means of quality control include measurement of nucleic acid quantity, purity, and fragment size of enriched nucleic acid sequences. Only libraries whose fragment sizes meet the requirements of sequencing instruments, with sufficient quantities and qualified purity can be used for subsequent sequencing. This quality control step is a part of the experimental process to ensure the experimental quality, wherein the controlling parameters are related to the choice of sequencing platform, but is not a necessary part of the technical scheme herein.

Sequencing: Perform sequencing according to the instrument manuals, device model, and instructions for reagents. The technology herein is compatible with NGS sequencing instruments manufactured by, but not limited to, Thermo Fisher, Illumina, BGI and other major suppliers, and all currently marketed instruments and reagents.

### Data Analysis Process

The logic of the data analysis process in this invention is to align (map) short sequence fragments (reads) obtained by NGS sequencing to all reference sequences in the microbial genome characteristic sequence database; Calculate the statistical characteristic metrics such as CV of the actual coverage of reads on each reference sequence. Compare the actual coverage of reads on each reference sequence with the theoretically predicted read coverage (if the microorganism represented by the reference sequence is present in the sample). The theoretically predicted read coverage herein can be constructed from mathematical models such as probabilistic and statistical models or from empirical/experimental data. The results of his comparison are reflected in several different metrics (such as NRMSE, etc.); Depending on whether the statistical characteristic metrics and the comparison representational metrics meet the required standards or not, the reference sequences that fail to meet the standards are eliminated. The screening operation is repeated until the statistical characteristic metrics and comparison representational metrics of all remaining reference sequences meet the pre-specified final criteria. Then the microbial species represented by these remaining reference sequences are the results of species identification. Calculate the proportion of the number of aligned reads for each of these reference sequences in the total number of reads aligned to reference sequences in the sample, that is, the proportion of microbial species represented by each reference sequence among all microorganisms identified in the sample.

The above reference sequence screening method based on the statistical characteristic metrics and comparison representational metrics of actual read coverage for each reference sequence is only one of the methods that can be used. Other suitable screening methods can also be used. For example, screening based on statistical characteristic metrics or comparison representational metrics, or enrichment analysis based on Fisher's exact test, or EM algorithm based on Bayesian probability, etc.

In this invention, all reference sequences in the database are clustered based on the sequence similarity between reference sequences, and a representative reference sequence, named seed, is selected for each cluster; Perform clustering operations on all seeds and select a representative reference sequence, named seed ID, for each cluster generated; Further clustering operations on seed IDs and selection of further seed IDs can be performed as needed. This operation can be performed as many times as necessary. In the end, a hierarchical clustering tree structure (with or without root) is constructed, in which the reference sequences of microbial genome characteristic sequence database constitute the leaves of the tree, forming the bottom tier of the tree structure. The node in the first tier above the leaf is the seed of the cluster in the first tier. And nodes in the other tiers are seed IDs of clusters formed by all nodes that are one tier below and connected to them. Screening of reference sequences in the database can start from the top node of the clustering tree and proceeds top-down tier by tier.

In some embodiments, said comparative analysis includes:
a) Remove low-quality reads and those containing sequencing adaptor sequences from said sequencing data;
b) The sequencing reads are aligned with the seed sequences of said characteristic sequence database. After removing duplicate reads generated by PCR amplification, perform statistical test for each seed sequence on independence between the events “there are reads aligned with reference sequences” and “reference sequences belong to the child clusters of all tiers except for the bottom one under this seed sequence”. All seed sequences of statistical significance form a set of primary screened seed sequences;
c) The sequencing reads are aligned with each of said primary screened seed sequences, wherein said primary screened seed sequences do not compete with each other for reads. Determine the read coverage and calculate the metrics characterizing coverage for each seed sequence. Select seed sequences based on their coverage metrics to form a set of secondary screened seed sequences;
d) Align sequencing reads with said secondary screened seed sequences, wherein seed sequences compete against each other for sequencing reads in the process. The read coverage metrics are calculated for each seed sequence, and the seed sequences that do not meet the requirements for read coverage metrics are removed iteratively. Seed sequences that remain after the alignment process are the tertiary screened seed sequences;
e) Merge all said tertiary screened seed sequences and perform sequence alignment between reference sequences represented by said tertiary screened seed sequences and sequencing reads. Obtain reference sequences that meet the requirements after an iterative screening process, wherein the threshold used in the screening process is more stringent than that used in step d);
f) Based on said reference sequences and their mapped sequencing reads obtained in step e), Calculate the total number of mapped reads and the relative abundance in reads at the species level. The total number of mapped reads for each species can be obtained by summing up the number of mapped reads from all reference sequences belonging to this species. And the relative abundance of each species can be calculated by dividing the total number of mapped reads for this species by the sum of total number of mapped reads of all species identified in this sample.

In some embodiments, wherein the sample to be tested is from a microbial host, step a) also includes removal of interference from the host nucleic acid sequencing data.

In some embodiments, in step d), a process of screening reference sequences within the cluster is also included following stepwise iterative removal of seed sequences that do not meet the requirements on read coverage:

Align the reference sequences of each cluster to which the seed belongs with the sequencing reads obtained in the previous step, wherein reference sequences within the same cluster compete for reads; Calculate the read coverage for each reference sequence. The reference sequences with poor read coverage were filtered out. The threshold for read coverage used in the screening iteration is more stringent than that described in step d).

In some embodiments, step g) is included after step f): exclude the interference of sequencing data from background contaminating species in the experimental environment.

In some embodiments, in step b), the statistical test on independence is Fisher's exact test, which specifically includes:

Reference sequences with more than a certain number of sequencing reads aligned are denoted as “has read aligned” or “no read aligned” otherwise. According to the hierarchical clustering relationship between seeds in the reference sequence database, statistical test is performed to determine whether a seed ID is significantly enriched with reference sequences denoted “has read aligned” in leaf nodes of each seed in the clustering tree under the seed ID. Seeds meeting the requirements are identified tier by tier in the clustering tree.

In some embodiments, the method for constructing said characteristic sequence database includes:
1) Obtain public databases containing reference sequences for said characteristic sequences. Remove terminal sequences of said reference sequences of said databases outside said amplification primers, and obtain the first database;
2) Sequences with ambiguous bases among said reference sequences in said first database are corrected based on intra-specific sequence similarity. And the redundant reference sequences with 100% similarity are removed based on species annotation and sequence similarity to obtain the second database;
3) Said reference sequences in said second database are clustered based on sequence similarity.

In some embodiments, while constructing said first database, remove terminal sequences and primer sequences outside said amplification primers of said reference sequences in said database.

In some embodiments, construction of said second database also includes:
1) Blast search NCBI NT/NR library for each reference sequence to obtain a set of matched reference sequences according to the rules of sequence similarity and/or coverage;
2) Identify the most representative taxonomic classification from the taxonomic annotations of said set of matched reference sequences, and use this taxonomic information to correct the species annotations of the reference sequences under consideration herein.

In some embodiments, said clustering includes a first clustering:
Cluster all non-redundant reference sequences based on sequence similarity; or
1) Cluster based on sequence similarity of non-redundant reference sequences within species;
2) Cluster the seeds obtained in 1) based on sequence similarity. Merge the seeds of different species in the same cluster together with their child sequences and re-cluster all entries based on 99.5% sequence similarity. Replace the old clusters involved in re-clustering with the newly formed clusters.

In some embodiments, said clustering also includes a second clustering:

In the case of having too many child sequences in the cluster of a seed, split the cluster obtained by said first clustering using a higher sequence similarity standard than that used by said first clustering. And the original cluster is replaced by the new clusters formed by splitting.

In some embodiments, said clustering also includes a third clustering:

Using different sequence similarity threshold, perform clustering on seed reference sequences of the clusters obtained by said second clustering to create a hierarchical tree.

In the following, the microbiological test based on NGS sequencing of prokaryotic 16S rRNA gene is taken as an example to explain the data analysis process in detail.

The data analysis process includes two major parts: 1) Methods for constructing 16s rRNA gene (rDNA) reference sequence database used in the analysis process; 2) Algorithms and work flows for sequencing data analysis.

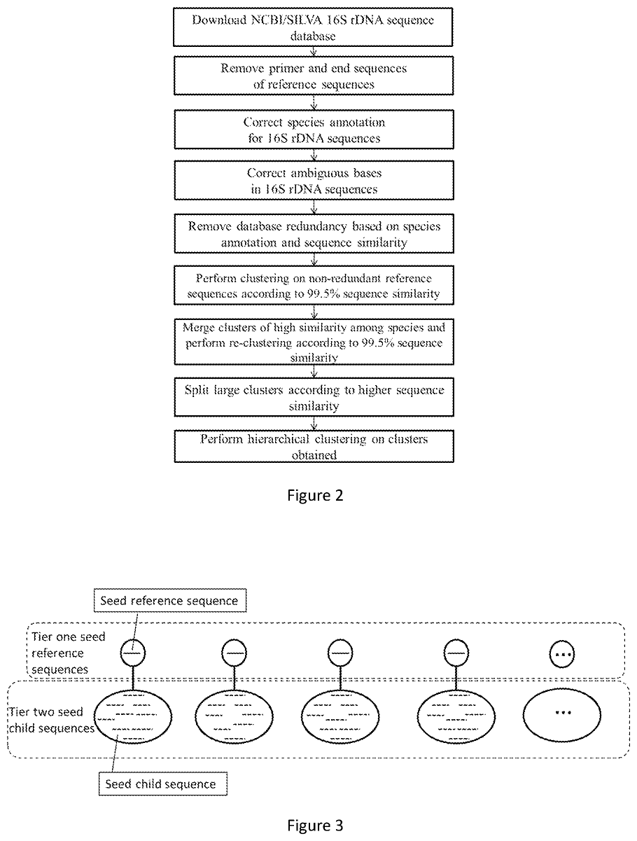

1. Construction of 16S rDNA reference sequence database. The schematic diagram of the main operating process is shown in FIG. 2.

1) Download the latest 16S rDNA reference sequence data from the public databases to ensure the completeness of sequence sources of the database. The downloaded databases include but are not limited to NCBI and SILVA database. The types and quantities of reference sequences contained in the database can be chosen according to the specific needs, for example, the database may comprise 100, or 250, or 500, or 1000, or 2000, or 10000, or even all existing types of reference sequences.

2) Remove the enrichment amplification primers and sequences located outside them from the reference sequences.

This step is aimed to obtain reference sequences that do not contain primers. The Smith-Waterman local alignment algorithm or other methods for alignment of short sequences can be used to match the amplification primers used for enrichment (referred to as amplification primers in this step) according to certain level of sequence similarities (such as but not limited to 80%, 85%, 90%, 95%, etc.) and locate enrichment amplification primers in the reference sequences. Then clip off amplification primers and sequences outside the primers at two ends according to the positions where primer sequences matches. Alternatively, remove the sequences outside both ends of primers only.

3) Correct species annotation of 16S rDNA sequences.

Since errors may exist in species annotation for some reference sequences in the database, this technical scheme performs corrections on the species annotation to ensure that the information in species annotation, to the greatest extent, reflects the correct species assignment to the annotated reference sequence. However, if the species annotation information of the reference sequence is sufficiently reliable, this correction may be omitted. Correction process: perform blast alignment against NCBI NT/NR library for each reference sequence to obtain a set of matched reference sequences according to the requirement of 99.5% sequence similarity and 99% sequence coverage (appropriate increase or reduction in similarity value is also within the scope of this technique). Choose the most representative species classification from the species annotation of the matched reference sequence set, and use this taxonomic information to correct the species annotation of the reference sequence under consideration. At the same time, based on the degree of uniqueness of the representative species classification in the species annotation of the matched reference sequence set, the certainty of the species annotation of the reference sequence is graded. The levels of certainty grading of the species annotation include: certain, limited and rare annotations (ordered from the most to the least certain). The number of certainty grading levels could be more or less or even omitted depending on the actual situation.

4) Correct ambiguous bases in 16S rDNA sequences.

Certain positions in some reference sequences in the database contain ambiguous or degenerate bases. In order to identify the most representative bases for these positions, rule-based corrections are performed. The main steps are: perform multiple sequence alignment using MUSCLE for reference sequences with greater than 97% similarity in the same species, and replace bases at the ambiguous base positions with the most representative bases. If there are still ambiguous bases present after this step, then perform multiple sequence alignment for reference sequences with greater than 97% similarity in the same genus and replace bases at the ambiguous base positions with the most representative bases. The ambiguous bases that remain after this process are retained.

5) Remove redundancy from the database based on species annotation and sequence similarity.

This step is to remove redundant reference sequences that are 100% identical to each other and ensure that the required reference sequences are not removed by mistake. Thus, this technical scheme requires that the reference sequences removed must be present within another or more reference sequences and their levels of certainty in taxonomic annotation are relatively low. In addition, the primer matching situation at both ends of reference sequences is also considered. For example, when a reference sequence with both ends matched with complete primer sequences but is enclosed completely by another reference sequence, retention of this reference sequence in the database is required; or if the certainty of taxonomic annotation of the shorter reference sequence is higher than the longer one, then the shorter reference sequence may be retained. There exist more complicated cases for handling, for example, a primer matched end of a reference sequence is contained by another longer reference sequence.

6) Perform clustering for non-redundant reference sequences within species using the specified threshold of sequence similarity.

The purposes of clustering are two folds. On one hand, clustering can diffuse the computational pressure of subsequent analyses, and on the other hand, reduce the competition among reference sequences during alignment, such that appropriate reference sequences are gradually identified more accurately. All non-redundant reference sequences within each species were clustered separately according to the standard of 99.5% sequence similarity (the standard of similarity could be adjusted appropriately according to the actual database reference sequence). After clustering, the representative sequence in each reference sequence cluster is chosen as the seed of the cluster, and the rest of the sequences in the same cluster are the child sequences of the seed.

7) Merge clusters with high species similarity and re-cluster reference sequences using specified similarity thresholds.

Perform clustering on all seeds obtained in 6) based on 99% sequence similarity (which can be adjusted appropriately based on the similarity used in seed clustering in 6)). Merge seeds and their child sequences of different species of the same cluster and perform re-clustering using 99.5% sequence similarity. Replace the old clusters involved in re-clustering with the newly obtained clusters. In addition, this step can be combined with 6) performing clustering with all sequences directly instead of clustering within species first and then merging similar clusters among different species as described above. However, if there are too many species close to each other in sequence similarity, then direct clustering method may not achieve good analytical results.

8) Split large-size clusters using higher sequence similarity.

When a seed has too many child sequences in a cluster, the cluster is split using higher sequence similarity (such as 99.6%, 99.8%, 99.9%, etc.) and replaced by the newly formed clusters after splitting. Thus, a database with bi-tiered cluster hierarchy is constructed: the first tier consists of seed reference sequences of clusters, and the second tier contains child reference sequences of all the clusters. The structure is shown in FIG. 3.

9) Hierarchical clustering of seed reference sequences of clusters constructed above.

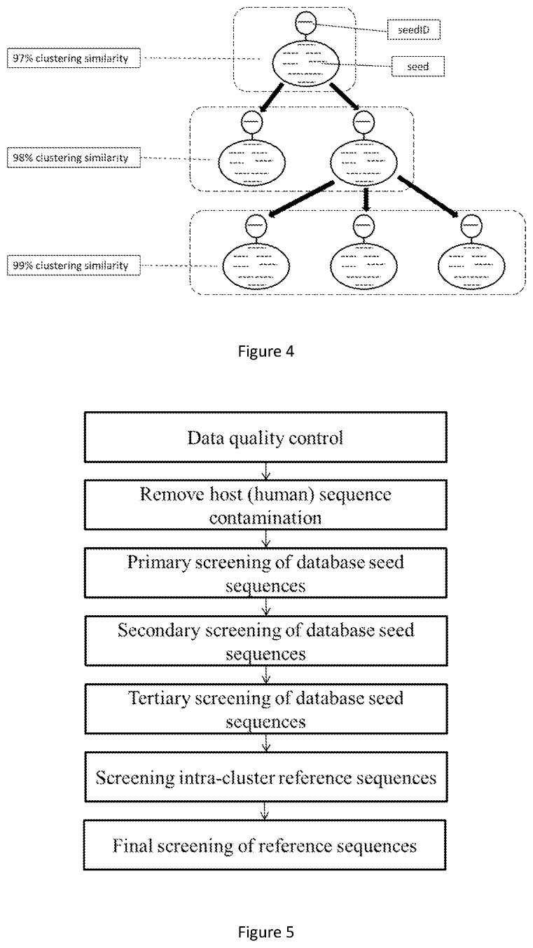

All seed reference sequences are clustered according to 97%, 98% and 99% sequence similarity (this three clustering similarity thresholds are used for representational purpose as long as tiered clustering can be achieved). The main steps are as follows: Firstly, all seed reference sequences are clustered according to 97% sequence similarity to obtain the first tier clusters (including seeds and their child sequences); And then, the reference sequences within each separate cluster are clustered according to 98% sequence similarity to obtain the second tier clusters; Subsequently, for each cluster of the second tier, the reference sequences within the cluster are clustered according to 99% sequence similarity to obtain the third tier clusters. Finally, an indexed hierarchical structure comprising all seed reference sequences in the database is constructed such that each seed reference sequence possesses seed IDs corresponding separately to clustering at three different sequence similarities. At this point, the index information of all reference sequences constitutes a four-column file. A schematic diagram of the seed clustering hierarchy for the reference sequence database is shown in FIG. 4.

2. The Algorithmic Process for Microbial Species Identification Using Sequencing Data. The Specific Workflow is Shown in FIG. 5.
1) Quality control of sequencing data.

Short read sequencing data in Fastq format are filtered upon entering the analytical pipeline. Low-quality reads and those containing adapter sequences are screened out. 2) Remove nucleic acid sequence contamination from the host (human).

Use bowtie2 (or other short read mapping tools) to align all read sequences with the human reference genome hg19 or hg38 with default parameters, filtering out reads that are aligned to the human genome sequences;

3) Primary screening of database seed sequences.

Reads are aligned with database seed sequences using hisat2 (no restriction on the choice of alignment tools as long as the tools are capable of achieving efficiently the goal of relatively accurate alignment between reads and reference sequences). According to the hierarchical clustering information, the primary screening of seeds is carried out using the enrichment analysis principle. The specific process is as follows:

All sequencing reads are aligned with all seed sequences in the database using hisat2 software. Filter alignment results with the condition that read mismatch rate is below a certain threshold (such as 0.5%, 1%, 1.5%, and 2%, etc.). Remove reads produced by PCR duplication from alignments. Each reference sequence aligned with more than a certain number of reads (such as 5, 10, 15, etc.) is denoted as “has read aligned” or “no read aligned” otherwise. Based on the clustering hierarchy of seeds in the reference sequence database, namely the clustering tree (see FIG. 4), perform enrichment analysis using Fisher's exact test. Perform statistical hypothesis testing for each seed in the clustering tree to examine if its leaf nodes are significantly enriched in “has read aligned” reference sequences, and identify seeds meeting the requirements tier by tier.

The enrichment analysis of Fisher's exact test is explained as follows:

Construct the following table for each seed and calculate the probability value of Fisher's exact test using the formula given below the table. The table shows the count of reference sequences that meet both row and column conditions. When the p value is less than or equal to a certain threshold (such as 0.001, 0.01, 0.05, 0.1, 0.2, 0.3, 0.4, 0.5, etc.), the clusters under the seed tested are significantly enriched with “has read aligned” reference sequences. And the smaller the p value is, the more significant the enrichment is.

| | A child | Not a | |
|---|---|---|---|
| | leaf of | child leaf | |
| | the seed | of the seed | Sum |
| Has read aligned | a | b | a + b |
| No read aligned | c | d | c + d |
| Sum | a + c | b + d | a + b + c + d |

$p = 1 - {\sum }_{i = 0}^{a - 1} \frac{( \begin{matrix}a + b \\ i\end{matrix} ) ( \begin{matrix}c + d \\ a + c - i\end{matrix} )}{( \begin{matrix}a + b + c + d \\ a + c\end{matrix} )}$

4) Secondary screening of database seed sequences.

Reads are aligned with primary screened seed sequences (no competition for reads among seed sequences) using bowtie2 (no restriction on the choice of alignment tools as long as the tools are capable of achieving the goal of relatively accurate alignment between reads and reference sequences. For example, other sequence alignment tools such as MAQ, SOAP, BWA, NovoAlign, etc.). Calculate the coverage of reads for each seed sequence. Calculate the metrics for seed sequence coverage and perform secondary screening of seed sequences accordingly. The specific computational process is as follows:

Align reads with the primary screened seed sequence using Bowtie2 (mainly to obtain alignments that meet certain sequence matching requirements. no competition for reads among reference sequences). Filter the alignments based on a threshold of read mismatch rate (such as 0.5%, 1%, 1.5%, or 2%, etc.), and remove reads produced by PCR duplication from the alignments. Calculate the coverage of reads for each seed sequence. Calculated metrics include CV (Coefficient of variation), coverage, mean depth (average sequencing depth), end gap (coverage gaps at one or both ends of a sequence), middle gap (coverage gaps in the middle of a sequence), etc. Screen the seed sequences using a relatively loose threshold of metric values, those of which meet the threshold requirements pass the secondary screening.

5) Tertiary screening of database seed sequences.

Seed sequences passing the secondary screening were grouped randomly. Within each group, sequencing reads are aligned with seed sequences using Bowtie2 or other sequence alignment tools such as MAQ, SOAP, BWA, NovoAlign and so on. Seed sequences compete for reads in the alignment operation. Calculate the read coverage metrics for each seed sequence alignment, and those of which fail to meet the requirement on the metrics of read coverage are removed iteratively. The detailed computational process is as follows:

The filtered seeds are randomly grouped and aligned with reads using Bowtie2. In the alignment process, seed sequences compete against each other for reads, which are randomly assigned among matched seed sequences with top ranking alignment scores. Then filter the alignments based on a threshold of read mismatch rate, and remove reads produced by PCR duplication from the alignments. Calculate coverage metrics for alignments, including Cor (Pearson's correlation coefficient which measures the level of agreement between the expected read coverage profile of a seed sequence predicted by the theoretical model and the actual coverage profile obtained by alignment), NRMSE (Normalized root mean square error, measures the level of difference in read coverage profiles between model prediction and actual alignment), read coverage, mean depth, end gap, middle gap, etc. Filter seed sequences depending on whether the read coverage metrics of a seed sequence alignment meet pre-specified standards such as mean depth>=15 or 20, end gap<=30 or 40, NRMSE<=0.4 or 0.35, middle gap<=0 or 5 or 10 or 15 and thereof; and mean depth herein is calculated in the scope of species meaning the sum of coverage depth of multiple seed sequences belonging to the same species must meet the requirement. Stringent standards lead to less seeds entering subsequent analyses. Seeds with good read coverage under stringent filtering standards are more likely to be selected as final candidate reference sequences. Filter out seed sequences that fail to meet the standards and continue the iteration until all remaining seed sequences meet the requirements. Merge seed sequences obtained from all groups and enter the next analysis process.

6) Screening reference sequences within clusters.

In previous steps, screening occurs in the seed tiers of the database. In this step, child sequences in each cluster represented by a seed identified through tertiary screening are screened in order to obtain reference sequences that meet the requirements more precisely. In addition, this step can also be adjusted to: if the read coverage of a seed sequence identified in step 5) is good enough, this seed sequence can become a candidate directly for the final step of screening. Only clusters represented by seeds with suboptimal read coverage were selected for within-cluster read alignment and screening of child sequences. The former method is used as an example for further elaboration in the following:

Read sequences are aligned with reference sequences of each cluster represented by a seed identified through tertiary screening (including the seed sequence and its child sequences) using Bowtie2 (no restriction on the choice of alignment tools as long as the tools are capable of achieving the goal of relatively accurate matching between reads and reference sequences). Reference sequences compete for reads during the alignment. Compute read coverage for each reference sequence which is filtered based on the coverage metrics. Reference sequences with poor read coverage are removed iteratively round by round. The detailed process is as follows:

Read sequences are aligned with reference sequences in each cluster represented by a seed identified through tertiary screening using Bowtie2. Alignment parameters used are more stringent than those used in previous steps, and competing for reads among reference sequences is allowed. In initial iterations, if reads are aligned to multiple reference sequences with the highest score (top1), they then are randomly assigned among these reference sequences. In subsequent iterations, the assignment of reads with top1 alignment score and thus shared by multiple reference sequences is conducted based on and proportionally to the read count and coverage metric values of each reference sequence achieves in the previous round of alignment. The higher the read count and the better the coverage metric values a reference sequence achieves, the higher the probability it acquires the read in the competition. After the above alignment is completed, filter alignments based on a more stringent threshold for reads mismatch rate. At sites of mismatch/indel in alignments, remove possibly mismatched reads based on the proportion among all aligned reads that the dominant allele occupies. Remove reads due to PCR duplication from alignments. Calculate read coverage metrics including Cor (calculate the level of agreement between the expected read coverage profile predicted by the theoretical model and the actual coverage profile obtained by alignment), NRMSE (calculate the level of difference in read coverage profiles between model prediction and actual alignment), coverage, mean depth, end gap, middle gap, etc. Depending on whether the read coverage metrics meet the pre-specified standards, filter out reference sequences that do not meet the standards. In each iteration, at most 1%, or 5%, or 10%, or 15%, or 30% (the proportion can be adjusted according to convergent rate needed) of the total number of reference sequences are filtered out until all remaining reference sequences meet the requirements. Then perform de-duplication on multiple reference sequences belonging to the same species: use MUSCLE (or other sequence alignment tools such as ClustalW, T-coffee, MAFFT, etc.) to perform pairwise alignment among reference sequences, and remove the one with poor read coverage from the two reference sequences that are identical in alignment position. Finally, the reference sequences identified from within-cluster screening are collected as candidate reference sequences for the next process.

7) Final screening of reference sequences.

Merge reference sequences obtained from within cluster screening, and align reads with reference sequences using Bowtie2 (or other sequence alignment tools such as MAQ, SOAP, BWA, NovoAlign and so on with reasonable parameter settings). Using more stringent parameters, reference sequences that finally meet the requirements are identified by iterative screening. The specific computing process is as follows:

Merge reference sequences obtained from within cluster screening, and align reads with reference sequences using Bowtie2. The alignment, screening, and iterative processes are the same as those in 6), but the screening parameters on read coverage are more stringent than those used in 6). After completing all computing in 6), reference sequences are filtered lastly based on the number of unique reads aligned to each reference sequence. If a reference sequence and another one are completely aligned in two regions each of which is specific to one of the two reference sequences (sequences at the end are not considered), depending on the sequence similarity and the number of unique reads possessed by each reference sequence, the reference sequence with less unique reads (the difference in the number of unique reads must meet certain requirements such as difference in multiples exceeding 1.5, 2, 2.5, etc.) is removed.

8) Calculate the proportion of microbial species in the sample.

Based on the read count of each reference sequence in the sample obtained in 7), calculate the read counts and relative proportions at the species level. Obtain the read count for a species by summing up the numbers of reads of all reference sequences belonging to this particular species. Then calculate the relative proportion of each species in the sample by dividing the read count of each species by the sum of read counts of all species present in the sample. Based on the statistics of each reference sequence (including mean depth, NRMSE, Cor, gap), calculate the confidence on the quality of result for each reference sequence using a logistic regression model. The training data for model construction are originated from analysis results of multiple runs of experiments.

9) Exclusion of contaminating background species.

Due to the presence of different types of environmental microorganisms in the sampling or experimental environment (including reagents and consumables used in the experiment), there exist background contamination in the microbial test results. The method used to remove background contamination in this technical scheme includes two steps: the first step, calculate the proportion in abundance of a species in the sample and determine whether this species is in the list of important clinical pathogens or not. If the proportion is very low and it is not present in the list of important clinical pathogens, then this species is ruled out as a false positive result; the second step, filter out species present in the negative control samples. However, due to the possibility that certain microbial species in the clinical samples may also be present in the environment, these species cannot be simply ruled out directly. To this end, multiple negative control samples were set up in the experiment, and an internal standard was introduced into each sample (including the tested sample and the control sample) to normalize the quantity of species in the same sample. The main method for removing background contaminating species is: calculate normalized read quantities of a species in clinical sample and control sample separately. Then the probability of the read quantity of a species as a result of contamination is calculated using the statistical distribution (e.g., Normal, Poisson, Weibull, or other known theoretical distributions or empirical distributions derived from re-sampling techniques such as bootstrapping or Jackknife and so on) of read quantity of this species in the negative control sample. If the probability is greater than a certain threshold (e.g. 0.001, 0.01, 0.05, 0.1, 0.2, 0.3, 0.4, 0.5, etc.), the species is considered background contamination and removed from the test result. Otherwise the species is retained in the test result.

By now, the process of screening reference sequence database based on sequencing reads identifies the final reference sequences that meet the requirements. According to the taxonomic annotation of species classification of the reference sequences, the species composition of microorganisms present in the sample is obtained.

### Microbial Sequencing Data Analysis Device, Computer Readable Storage Medium and Electronic Device

This invention also relates to a microbial sequencing data analysis device, said device comprises:
Sequencing data acquisition module, which is used to obtain sequencing data, said sequencing data is obtained by targeted enrichment of microbial characteristic nucleic acid sequences followed by sequencing the enriched products using next generation sequencing technology;
Characteristic sequence database construction module, which is used to perform clustering analysis on said characteristic sequences to obtain the characteristic sequence database, said clustering analysis is defined as said clustering analysis in the method described above;
Comparative analysis module, which is used to carry out alignment analysis between said sequencing data and said characteristic sequence database to identify microbial composition of said tested sample, said alignment analysis is defined as said alignment analysis in the method described above.

In an embodiment, said device comprises:
Sequencing data acquisition module, which is used to obtain sequencing data, said sequencing data is obtained by targeted enrichment of microbial characteristic nucleic acid sequences using PCR primers followed by sequencing the enriched products using next generation sequencing technology;
Characteristic sequence database construction module, which is used to perform clustering analysis on said characteristic sequences to obtain the characteristic sequence database, said characteristic sequence database contains one or more tiers of clusters. Each tier of cluster has at least one child seed, and the bottom-tier cluster has several seeds as reference sequences;
Comparative analysis module, which is used to carry out alignment analysis between said sequencing data and said characteristic sequence database to identify microbial composition of said tested sample.

The Comparative analysis module includes:
a) The first module, which is used to remove low-quality reads and reads containing sequencing adaptor sequences from the sequencing data;
b) The second module, which is used to align read sequences with the seed sequences of said characteristic sequence database, remove reads generated by PCR duplication, perform statistical independence tests on the presence of read alignments and seed sequences, and identify the seed sequences correlated with the presence of read alignments as the primary screened seed sequences;
c) The third module, which is used to align read sequences with said primary screened seed sequences, wherein said primary screened seed sequences do not compete for reads, calculate the coverage of reads for each seed sequence, calculate and evaluate the coverage metrics for each seed sequence based on which conduct secondary screening of seed sequences;
d) The fourth module, which is used to align read sequences with seed sequences obtained by secondary screening, wherein seed sequences compete for reads in the process of alignment, calculate read coverage metrics for seed sequences during the alignment, and remove seed sequences whose read coverage metrics does not meet the requirements gradually through iterations, and hence obtain the tertiary screened seed sequences;
e) The fifth module, which is used to merge said tertiary screened seed sequences and align reads with them, obtain reference sequences meeting the requirements through stepwise iterative screening, wherein the threshold used in stepwise iterative screening is more stringent than that in step d);
f) The sixth module, which is used to calculate the read counts at the species level and the proportion of read counts for each species in the total read counts based on said reference sequences and their read counts obtained in step e); During the calculation, sum up the read counts of multiple reference sequences belonging to the same species to obtain the read count for this particular species. Then calculate the proportion of each species in the sample by dividing the read count for each species with the total read count of all species present in the sample.

This invention also relates to a computer readable storage medium. Said computer readable storage medium is used to store computer instructions, programs, code sets or instruction sets that, when being executed on a computer, allow the computer perform step ii) of the method described above.

Any combination of one or more computer readable media may be used. Computer readable media can be computer readable signal media or computer readable storage media. A computer readable storage medium may be, for example, but not limited to an electrical, magnetic, optical, electromagnetic, infrared, or semiconductor system, instrument or device, or any combination of the above. More specific examples (non-exhaustive list) of computer readable storage media include: electrical wiring of one or more electrical connections, portable computer disk, hard disk, random access memory (RAM), read-only memory (ROM), erasable programmable read-only memory (EPROM or flash), optical fiber, portable compact disk read only memory (ROM), optical storage device, magnetic storage device, or any appropriate combination of the above. For purposes of this document, a computer readable storage medium may be any tangible medium containing or storing a program that may be used by or in conjunction with an instruction execution system, instrument, or device.

A computer readable signal medium may include data signals carrying computer readable program code propagated in baseband or as part of a carrier. Said transmitted data signals may take many forms including, but are not limited to, electromagnetic signals, optical signals, or any suitable combination of the above. A computer readable signal medium may also be any computer readable medium other than a computer readable storage medium that can emit, propagate, or transmit programs for use by or in conjunction with an instruction execution system, instrument, or device.

The program code contained on a computer readable medium may be transmitted in any appropriate medium including, but not limited to, wireless, wire, fiber optic cable, RF, etc., or any suitable combination of the above.

The computer program code used to perform this open operation can be written in one or more programming languages or a combination thereof. Programming languages include object-oriented programming languages such as Java, Smalltalk, C++, as well as regular procedural programming languages such as “C” or similar programming languages. The program code can be executed entirely on the user's computer, partially on the user's computer, as a standalone software package, partially on the user's computer and partially on a remote computer, or completely on a remote computer or server. In cases involving remote computers, the remote computer can be connected to the user's computer through any kind of network, including a local area network (LAN) or wide area network (WAN), or it can be connected to an external computer (for example, through the Internet using an Internet service provider).

According to another aspect of the invention, this invention also relates to an electronic device, including:
i) One or more processors; and
ii) A storage device that stores one or more programs, when one or more programs are executed by said one or more processors such that said one or more processors implement step ii) of the method described above.

Optionally, electronic devices may also include transceivers. A processor is connected to a transceiver by such as a bus. It should be noted that the transceiver is not limited to one in actual applications, and the structure of the electronic device does not constitute restrictions to this application embodiment.

Herein the processor can be CPU, general purpose processor, DSP, ASIC, FPGA or other programmable logic devices, transistor logic devices, hardware components or any combination thereof. It may implement or execute the various exemplary logic boxes, modules and circuits described in conjunction with the contents disclosed in this application. The processor can also be a combination of computing functions, such as a combination of one or more microprocessors, DSP and microprocessors, etc.

A bus may include a pathway to transmit information between components described above. The bus can be PCI bus, EISA bus, etc. Bus can be divided into address bus, data bus, control bus and so on. Memory can be ROM or another type of static storage device that stores static information and instructions, RAM or another type of dynamic storage device that stores information and instructions. It can also be EEPROM, CD-ROM, or other CD storage, optical disk storage (including compression disc, laser disc, CD, digital general disc, blue-ray disc, etc.), disk storage medium or other magnetic storage devices, or can be any other media, capable of being used to carry or store desired program codes in the form of instruction or data structure and accessible by computers, but not limited to this.

In the following sections, embodiment of the present invention will be illustrated in detail using examples.

### Example 1: Database Construction

This example takes 16s rDNA as an example to illustrate the process of reference sequence database construction.

1) Download the latest 16S rDNA reference sequence data from the public databases to ensure the completeness of sequence sources in the database. The downloaded databases include but are not limited to NCBI and SILVA databases. The types and quantities of reference sequences included in the database can be determined depending on the specific needs, for example, 100, 250, 500, 1000, 2000, 10000 species, even all existing types, etc.
2) Remove enrichment amplification primers and the sequences external to the two primers in the reference sequence.

The goal of this step is to obtain a reference sequence that does not contain primers. The Smith-Waterman local alignment algorithm or other methods for aligning and positioning short sequences can be used to match and locate the amplification primers used for enrichment (referred to as amplification primers in this step) in reference sequences according to certain sequence similarities (such as but not limited to 80%, 85%, 90%, 95%, etc.). Then according to the positions of primer matching, remove the amplification primers and the terminal sequences external to the primers. Alternatively, only the terminal sequences external to the primers may be removed.

3) Correct species annotation for 16S rDNA sequences.

Since errors in species annotation may exist for some reference sequences in the database, this technical scheme corrects the species annotation to ensure that species annotations reflect the correct species assignments to the greatest extent. However, if the species annotation of the reference sequence is sufficiently reliable, this correction may be omitted. Correction process: perform blast alignment against NCBI NT/NR library for each reference sequence to identify a set of matched reference sequences satisfying the requirement of 99.5% sequence similarity and 99% sequence coverage (appropriate increase or reduction in similarity requirement is also within the scope of this technique). Select the most representative species classification from the species annotation of the matched reference sequence set, and use the selected species classification to correct the species annotation of the subject reference sequence. At the same time, based on the degree of dominance of the representative species classification among the species annotations of the matched reference sequence set, the certainty level of the species annotation of the subject reference sequence is graded. The certainty levels of species annotation include: centain, limited centainty, rare annotation (rank ordered from high to low by certainty). The number of levels in certainty can also be more/less than described above or even no grading at all according to the actual situation of certainty in annotation.

4) Correct ambiguous bases in 16S rDNA sequences.

Some positions in reference sequences in the database are ambiguous or degenerate bases. In order to determine the most representative bases in these positions, rule-based correction is performed. The main steps are: perform multiple sequence alignment using MUSCLE for reference sequences of sequence similarity greater than 97%, or 97.5%, or 98%, or 98.5% within the same species, and replace the ambiguous bases with the most representative bases. If there still exist ambiguous bases after this step, perform multiple sequence alignment for reference sequences with 97%, or 97.5%, or 98%, or 98.5% sequence similarity in the same genus, and replace the ambiguous bases with the most representative bases. The ambiguous bases that remain after processing are retained.

5) Remove redundancy from the database according to species annotation and sequence similarity.

This step is to remove the redundant reference sequences that are 100% similar to each other and ensure that the needed reference sequences are not removed by mistake. Therefore, this technical scheme requires that the removed reference sequences must be included by other reference sequences, and that the species annotation of the removed reference sequences has a low level of certainty.

6) Perform clustering analysis on non-redundant reference sequences within species according to specified thresholds of sequence similarity.

Perform clustering analysis on all non-redundant reference sequences within each species according to sequence similarity criteria of 98%, or 98.5%, or 99%, or 99.5% (sequence similarity criteria may be adjusted appropriately according to the actual situation in database reference sequences). After clustering analysis, the representative sequence in each reference sequence cluster serves as the seed of the cluster, and the rest of the reference sequences in the same cluster become its child sequences.

7) Merge clusters with high degree of inter-species sequence similarity and conduct clustering on merged cluster members using specified threshold of sequence similarity.

Perform clustering on all the seeds obtained in 6) according to 98%, or 98.5%, or 99% (which may be adjusted appropriately based on the sequence similarity used in seed clustering in 6)) sequence similarity. Merge seeds of different species in the same cluster and their child sequences, and then re-cluster them according to 99.5% sequence similarity. Replace the old clusters participating in the re-clustering with the newly formed clusters. In addition, this step may be combined with 6) to perform clustering on all sequences directly instead of performing within-species clustering first and then merging clusters of similar sequences between species as described above. However, if too many species are close to each other, the direct clustering method may not be able to achieve ideal analytical effects.

8) Split clusters of large sizes using higher sequence similarity.

In the case that there are too many seed child sequences in a cluster, the cluster is split according to the criteria of higher sequence similarity (such as 99.6%, 99.8%, 99.9%, etc.), and newly formed clusters as a result of splitting replace the clusters before splitting. At this point, a database of two-tier clustering hierarchy is formed: the first tier consists of seed reference sequences of the clusters, and the second tier comprises child reference sequences of all the clusters.

9) Perform hierarchical clustering on seed reference sequences of the constructed clusters.

Perform clustering on all seed reference sequences using sequence similarities of 97%, 98% and 99% sequentially (Only these three clustering similarity criteria are taken as representative examples, as long as the result of tier by tier clustering is achieved). The main steps are: Firstly, perform clustering on all seed reference sequences according to 97% similarity criterion to obtain first tier clusters (including seed and its child sequences); Then perform clustering on reference sequences within each cluster separately using 98% similarity criterion to obtain the second tier clusters; Subsequently, perform clustering on reference sequences within each cluster of the second tier separately using the 99% similarity criterion to obtain the third tier clusters. In the end, an indexed hierarchical structure containing all seed reference sequences in the database is constructed, such that each seed reference sequence obtains seed IDs corresponding to clusters of three different sequence similarity respectively. By now, the indexing information for all reference sequences constitutes a file consisting of four columns of information. The clustering hierarchy of seeds of the reference sequence database is shown in the figure below.

### Example 2: In Silico Simulation and Data Analysis

### Generation of Simulated Data

Step 1). A single or different combination of multiple reference sequences was selected arbitrarily from the database to generate simulated samples. The selected reference sequences were fragmented by random disruption to generate simulated reads with length distribution following normal distribution. Sets of simulated reads with specified sequencing depth were randomly generated to simulate sequencing results. A total of 83 samples consisting of different species mixed equally in quantity were generated by simulation, including 24 samples containing single species and 59 samples containing a mixture of multiple species (ranging from 2 to 12). The average depth of simulated reads ranged from 20× to 800×.

### Analysis Process

Database: A database containing 2119 pathogenic bacterial species was used for the analysis of simulated data. The hierarchical relationships between reference sequences were constructed according to 97%, 98% and 99% sequence similarity. The database included a total number of 83,886 reference sequences, among which a total number of 34,025 representative sequences in the first tier (Redundancy in reference sequences in this version of database was removed).

Step 2). Due to the large number of seeds in seed tiers in this database, Hisat2 software was used to perform alignment between sequencing reads and database seed reference sequences using the parameter—score-min L,−1,−0.08—no-spliced-alignment—no-templatelen-adjustment—seondary-k 100000—mm—no-softclip. The alignment was filtered according to 1% mismatch reads rate, and the reads generated by PCR duplication in the alignment were removed. Then, the number of aligned reads for each reference sequence was counted, and labeled the reference sequences that have more than 10 reads aligned as “has read aligned” otherwise as “no read aligned”. And then, using the enrichment analysis method based on the clustering hierarchical relationship, seed IDs whose child nodes were significantly enriched with reads were selected according to the relatively loose enrichment p value of 0.1. The corresponding seeds were extracted as candidate reference sequences for the next step.

Step 3). Sequencing reads were aligned with candidate reference sequences obtained in step 2) using Bowtie2 with parameter setting—min-score L,−1,−0.1−a, wherein reference sequences did not compete for reads. Then, the alignments were filtered according to 0.5% mismatch reads rate, and reads in the alignment produced by PCR duplication were removed. Read coverage for each reference sequence was calculated, and the reference sequence was filtered using parameter CV<=0.5 & gap<=40 & mean depth>=20. The reference sequences passing the screening threshold became candidate sequences for the next step.

Step 4). Sequencing reads were aligned with candidate reference sequences obtained from screening in step 3) using Bowtie2 software with the same parameter setting as in 3). Then the alignments were filtered using 0.5% mismatch reads rate, and reads in the alignments generated by PCR duplication were removed. Metrics such as CV, gap, Cor, CV/Cor and mean depth for each reference sequence were calculated. Reference sequences were filtered iteratively according to the criteria CV<=0.55 & Cor>=0.6 & mean depth>=20 & gap<=40 until all reference sequences left meet the requirements.

Step 5). The reference sequences obtained in step 4) was further screened. If the read coverage of a reference sequence met the criteria CV<=0.4 & Cor>=0.9 & gap==0, then it entered into the final iteration directly; Otherwise, iterative screening was performed inside the second-tier cluster of the reference sequences wherein reference sequences inside the cluster competed for reads. The steps of internal iterative screening inside the second-tier cluster are the same as 3) and 4), wherein the threshold parameter for the step equivalent to step 4) was CV<=0.55 & Cor>=0.6 & mean depth>=20 & gap<=25. Reference sequences meeting the criteria entered in the next step.

Step 6). All reference sequences obtained in step 5) that met the requirements were combined to carry out the final iterative screening. Alignment with sequencing reads was performed using Bowtie2 with the same parameter setting as in step 4). Mismatch reads rate of 0.5% was used to filter the alignments, and reads generated due to PCR duplication were removed from the alignments. At sites in a reference sequence alignment where the proportion of the dominant allele was lower than 0.95, only reads with allele in agreement with the reference sequence were retained. The read coverage for each reference sequence was calculated, based on which reference sequences were screened iteratively using threshold CV<=0.55 & Cor>=0.6 & mean depth>=20 & gap<=25. The reference sequence with coverage farthest away from meeting the threshold requirement was eliminated in each iteration of the screening until all remaining reference sequences met the threshold requirement. Then sequence alignment was performed for all possible pairs of reference sequences in each species. The reference sequences that ultimately met the requirements were the final target sequences, and the corresponding annotated species was the target species. In addition, the total read count for each species and its relative proportion in the sample were calculated.

### Analysis Results

The analysis of the simulated experimental data showed that the average sensitivity and precision of the test for species in the sample reached 98.65% and 98.79% respectively, wherein, the sensitivity and precision for samples comprising single species were both 100%.

### Example 3

In this example, Gram-positive bacteria were used as experimental subjects, which were a pure strain of known species cultured in liquid medium. The purpose is to investigate whether this technical scheme is capable of identifying correctly the microorganisms in the samples, and to investigate the limit of sensitivity in detection for this technical scheme.

### 1. Sample Preparation

Experimental samples of single species, Staphylococcus epidermidis, were cultured on agar plates in laboratory and colonies were counted to confirm the number of bacterial CFU added to the samples. 2560 CFU of bacteria were added to each of samples INQ19M0101 and INQ19M0102; 640 CFU were added to INQ19M0103 and INQ19M0104; 160 CFU were added to INQ19M0105 and INQ19M0106; 40 CFU were added to INQ19M0107 and INQ19M0108; And 10 CFU were added to INQ19M0109 and INQ19M0110.

### 2. Experimental Process

1. Sample DNA extraction. Ezup Bacterial Genomic DNA Extraction kit (Sangon Bioengineering (Shanghai) Co., LTD., Item number B518255-0100) was used to extract sample DNA by following the kit instructions.

In this example, the tested sample is bacteria, and the extraction scheme is to extract DNA from the sample. In this technical scheme, the nucleic acids extracted may also be RNA, or DNA and RNA at the same time; Nucleic acid extraction kits used are not limited to this particular manufacturer or this item number.

2. Target gene amplification. PCR amplification primers: F: 5′AGAGTTTGATCMTGGCTCAG3′ (SEQ ID NO: 1), or 5′AGAGTTTGATCCTGGCTCAG3′ (SEQ ID NO: 2), or 5′CTCCTACGGGAGGCAGCA3′ (SEQ ID NO: 3), or 5′GTGCCAGCMGCCGCGG3′ (SEQ ID NO: 4), or 5′AAACTYAAAKGAATTGACGG3′ (SEQ ID NO: 5), or 5′GCAACGAGCGCAACCC3′ (SEQ ID NO: 6), or 5′AGAGTTTGATCATGGCTCAG3′ (SEQ ID NO: 7), or 5′TAACTGAAGAGTTTGATCCTGGCTC3′ (SEQ ID NO: 8); R: 5′GGTTACCTTGTTACGACTT3′ (SEQ ID NO: 9), or 5′GGYTACCTTGTTACGACACTT3′ (SEQ ID NO: 10), or 5′CTGCTGCSYCCCGTAG3′ (SEQ ID NO: 11), or 5′GWATTAACCGCGGCKGCTGTG3′ (SEQ ID NO: 12), or 5′CCGTCAATTCMTTTRAGTTT3′ (SEQ ID NO: 13), or 5′GGGTTGCGCTCGTTG3′ (SEQ ID NO: 14), or 5′AAGGAGGTGWTCCARCC3′ (SEQ ID NO: 15), or 5′TAGGGTTACCTTGTTACGACACTT3′ (SEQ ID NO: 16) or 5′TACGGTTACCTTGTTACGACACTT3′ (SEQ ID NO: 17). The reagent used was Ex Taq HS manufactured by Baoriyi Biotechnology (Beijing) Co., LTD., Item number RR006A. In this embodiment, the method used to enrich target sequences is PCR enrichment of 16S rRNA gene sequences. In this technical scheme, the enrichment method may also be hybridization capturing; the enzyme used for PCR reaction is not limited to this item number of this manufacturer. It may be another item number or another manufacturer's Taq enzyme, or other types of enzyme, as long as it is capable of amplifying the DNA sequences; for prokaryotes such as bacteria, the 16S rRNA gene sequence is chosen as the target sequence in this embodiment. However, there are many options for the choice of target sequence, including but not limited to the sequences in regions such as 16S/18S/ITS, which can be chosen and combined arbitrarily according to the purpose of sequencing. Even for prokaryotes, the target sequences are not limited to 16S, nor are the amplification primers limited to the sequences listed in this example; The reaction system and amplification conditions listed in this example are only appropriate reaction conditions for the Taq enzymes and primers used, and shall not be regarded as limitations of this technical scheme.

The contents of the electronic sequence listing (US18019995_07082025_Sequence-listings_VP143093-010100_ST25; Size: 7 k bytes; and Date of Creation: Jul. 8, 2025) are herein incorporated by reference in its entirety.

### Reaction System:

| Component | Volume(μl) |
|---|---|
| 10*buffer | 7.5 |
| dNTPs | 6 |
| Ex Taq | 0.375 |
| Primer F | 3 |
| Primer R | 3 |
| Template amount | 10 |
| ddH₂O | 45.125 |

### Reaction Condition:

| | Temperature | | Time | |
|---|---|---|---|---|
| 94° C. | | | 3 | min |
| | 30 cycles | 94° C. | 30 | sec |
| | | 55° C. | 30 | sec |

| | |
|---|---|
| 72° C. | 1 min 30 sec |

| | | |
|---|---|---|
| 72° C. | 7 | min |

| | |
|---|---|
| 4° C. | hold |

3. Product purification. The main reagent used is DNA screening magnetic beads (Wuxi biomag biotechnology Co., LTD., BMSX). The operating procedures are as follows: in this example, the PCR product is purified using the DNA screening magnetic beads. In this technical scheme, the purification method is not limited to the use of magnetic beads, and may be other methods of PCR product purification such as affinity column purification; And magnetic beads used for purification is not limited to DNA binding magnetic beads, and is not limited to the product of this company, as long as the magnetic beads used are capable of purifying PCR products; Moreover, weather purification is required or not, or the choice of purification approach, is dependent upon the approach of target sequence enrichment used in the last step and the choice of reagents and reaction conditions for the reaction used in the next step. In addition, purification may not be needed, or may not purify “PCR products”.
a) Magnetic beads were kept at room temperature for 30 minutes and then vortexed for 3 minutes to mix thoroughly. 37.5 μl of magnetic beads was added into the well of a 96-well plate.
b) After PCR reaction was completed, PCR tubes were removed from the equipment and centrifuged for 10s. PCR products were transferred into the corresponding wells of the 96-well plate prepared in the last step. The plate was sealed with film and vortexed for 30s. After standing still for 5 min, the plate was centrifuged transiently and placed on magnetic plate for 5 min. Supernatant was carefully removed.
c) Added 180 μl of the newly prepared 75% ethanol. Held the bottom of the magnetic plate, move the 96-well plate horizontally to the adjacent slot of the magnetic plate and back for 3-5 times, and let it stand still for 5s. Removed supernatant after the liquid in wells became clear.
d) Repeat step c).
e) Removed the 96-well plate from the magnetic plate, performed brief centrifugation, and put it back on the magnetic plate. Removed the remaining ethanol using 100 μl pipette, and opened the cover to dry until the pellet of magnetic beads appeared slight cracked.
f) Added 22 μl ddH₂O, sealed the plate with film, vortexed the plate for 30s, let it stand still for 5 min, and then centrifuged transiently. The 96-well plate was placed on the magnetic plate and stood still until the liquid inside became clear. And all the supernatant was transferred to a new 1.5 mL low adsorption centrifugal tube, which was the purified product.

4. Quantification of PCR product. Purified PCR products were quantified using Qubit 3.0 assay kit (Qubit dsDNA BR Assay Kit, Thermo Fisher Scientific Q32850) and following the instructions for reagents and instruments. In this example, PCR products were quantified using a fluorescent dye method. In this technical scheme, for nucleic acids resulted from targeted enrichment, quantification methods are not limited to fluorescent dye method, but could also be other dye methods or non-dye methods such as ultraviolet spectrophotometry, quantitative fluorescence PCR, capillary electrophoresis or microfluidic electrophoresis combined with nucleic acid dye fluorescence imaging method, etc.; For fluorescent dye method, the assay kit used is not limited to this manufacturer or this item number; A method would be acceptable as long as it can measure accurately the mass (referring to the amount of substance, not quality) of nucleic acids in the sample; If ultraviolet spectrophotometry is used, nucleic acid quality information can be obtained, which also belongs to the scope of quality control in this technical scheme, and does not constitute an innovation of this technical scheme.

Fragmentation of target gene. In this example, deoxyribonuclease I (DNase I) (manufactured by New England Biolabs, Item number M0303S) was used to fragment the nucleic acids tested. The operating procedure is as follows: In this embodiment, nuclease DNase I is used to fragment the nucleic acids in the sample. In this technical scheme, the method used is not limited to the use of biological methods such as enzymes, can also be physical methods such as ultrasound, chemical methods, or other types of methods. It can be controlled to break long nucleic acid fragments into short fragments without introducing other changes that will affect the recognition of nucleic acid sequences; Even if biological methods are used, they are not limited to DNase I, can also be other enzymes that are able to cut DNA, such as other DNA endonucleases, DNA transposase (such as Tn5 transposase), CRISPR, etc.; The use of DNase I enzyme is not limited to this manufacturer or this item number; The reaction conditions adopted are only purported to be compatible with the reagent used in this embodiment. The reaction conditions may change with the changes in reagents and methods, and do not constitute a limitation of the technical scheme. In this embodiment, 5-100 ng of nucleic acids obtained in the previous step were added to the reaction. In this technical scheme, 5-100 ng is not a limiting condition, but only a more suitable condition. More or less nucleic acids can also be used in this technical scheme. Using this technical scheme with 0.01 ng or less of nucleic acids can also produce correct results. In this technical scheme, the amount of DNase I used is only a relatively appropriate amount under the current conditions, and is not a limiting factor. In this technical scheme, the description for the purification method is the same as before.
a) Removed the purified amplification product of 16S rRNA gene of the sample, enzyme buffer, EDTA and MnCl₂ from the refrigerator, melt, shook, and centrifuge transiently, then set aside.
b) Placed PCR tubes corresponding to the number of samples on the ice box, added X μL of 60 ng purified 16S rRNA gene amplification products and water (30-X) μl at the same time. EDTA was diluted by a ratio of 1:4 for later use (final concentration: 1.25 mmol/L).
c) Diluted the original enzyme solution (2 U/μl) to 0.1 U/μl: Placed a 0.2 ml PCR tube on ice box, added 19 μl of 1× DNase I buffer (diluted from 10× buffer). Removed DNase I enzyme from the refrigerator followed by brief centrifugation, took I μl of the enzyme and added it into the PCR tube. Mixed the tube content by point vortexing for 30-40 times, and placed the tube on ice box immediately after brief centrifugation.
d) Preparation of enzyme, enzyme buffer and MnCl₂ MIX: cut 30 ng of DNA with 0.01 U of enzyme:
   i. Placed a 1.5 ml centrifuge tube on ice box. Added sequentially ((n+2)*2) μl of 10× DNase I buffer, ((n+2)*2) μl of MnCl₂, and ((n+2)*0.1) μl of diluted DNase I enzyme (0.1 U/μl) into the tube. Shook the tube 40-50 times by point vortexing and placed it on ice box immediately after brief centrifugation.
   ii. Added 4.1 μl of MIX into each well of an 8-well PCR tube strip and took 15 μl of solution prepared in b) using an 8-channel pipette and added to the corresponding wells. Covered the tubes and shook 5-10 times by point vortexing, then placed on ice box immediately after brief centrifugation.
e) Incubated the 8-well tube strip on a PCR machine for 15 min at 20° C.
f) Took out the 8-well tube strip after the incubation and added 5 μl of diluted EDTA to each well to terminate the reaction. Shook the tube strip followed by brief centrifugation. The reaction products were fragments of variable sizes. Proceeded immediately to the next step of magnetic bead purification.

6. Purification of fragmentation products. The main reagent used was DNA binding magnetic beads (Wuxi Biomeag Biotechnology Co., LTD., BMSX). The operating procedure is as follows:
a) Vortexed for 3 min magnetic beads equilibrated at the room temperature for 30 min, mixed thoroughly, and added 50 μl into each well of a 96-well plate with well labels.
b) Transferred the enzyme digestion products to the corresponding wells of the 96-well plate prepared in the previous step. Sealed the plate with film and vortexed it for 30 s. Let the plate sit for 5 min and centrifuged briefly. Placed it on a magnetic plate for 5 min, then remove supernatant carefully.
c) Added 180 μl of newly prepared 75% ethanol into each well, held the bottom of the magnetic plate, moved the 96-well plate horizontally to the adjacent slot of the magnetic plate and back for 3-5 times, and let it stand for 5 s until the liquid became clear.
d) Repeat step c).
e) Removed the 96-well plate from the magnetic plate, centrifuged the plate briefly, and place it back onto the magnetic plate. Removed the remaining ethanol using 100-μl pipette, and opened the cover of the 96-well plate until the pellet of magnetic beads appeared slightly cracked.
f) Added 42 μl TE buffer to each well, sealed the plate with film, and vortexed it for 30 s. Let the plate sit for 5 min, then centrifuged it briefly. Placed the 96-well plate on the magnetic plate and let it sit until liquid inside became clear. Drew 40.2 μl of supernatant from each well as purified products and transferred to a new 96-well plate.

7. DNA end repair. The reagents used include Pfu enzyme (Tiangen Biochemical Technology (Beijing) Co., LTD., EP101), dNTP Mixture (Tiangen Biochemical Technology (Beijing) Co., LTD., CD111), and DNA binding magnetic beads (Wuxi biomeag Biotechnology Co., LTD., BMSX). The operating procedure is as follows: In this embodiment, the dsDNA after terminal repair has blunt ends, meaning the ends of both strands of the DNA are aligned. In this technical scheme, the end repair of small fragments varies according to the sequencing platform selected. For example, when Illumina sequencing platform is used, the end of a strand of repaired dsDNA will have a protruding adenine (A), and the repair method does not constitute a limitation of this technical scheme; The enzyme used for terminal repair is not limited to Pfu enzyme, may also be Taq enzyme or other enzymes; Pfu enzyme is not limited to the item number of the manufacturer herein. In this embodiment, fragment screening based on length is performed following the terminal repair step. In this technical scheme, fragment screening is not limited to the use of DNA fragment screening magnetic beads, it may also be other methods such as recovery of nucleic acid fragments of required length following electrophoresis as long as selection of nucleic acid fragments is feasible; The DNA fragment screening magnetic beads are not limited to the manufacturer or the item number herein; The length of fragments retained after screening is related to the sequencing instrument, sequencing reagent and sequencing parameter setting selected, and does not constitute a limitation of this technical scheme; The timing of length based screening of nucleic acid fragments is also not limited to after terminal repair, it may also be before terminal repair, or after the next step, or after the step after the next step of the experiment. This adjustment does not constitute a limitation of this technical scheme. In this technical scheme, the description of purification method is the same as before.
a) Reagents were added successively into a low adsorption tube containing 40 μl of DNA, and then mixed thoroughly by repeated blow-out and aspiration using a pipette. Vortexed the tube followed by brief centrifugation, and placed it at 72° C. for 15 min on a PCR instrument.

| Component | Volume(μl) |
|---|---|
| DNA | 40.2 |
| 10X Pfu Buffer | 5 |
| dNTPs(2.5 mM) | 4 |
| Pfu | 0.8 |
| Total system | 50 |

b) The reaction products were added to wells of a 96-well plate, each of which was preloaded with 32.5 μl of magnetic beads, then sealed the plate with film. Vortexed the plate for 30 sec, then let it sit for 5 min followed by brief centrifugation. Placed the plate on magnetic plate for 5 min and transferred supernatant to the corresponding wells of new 96-well plate preloaded with 10 μl of magnetic beads. Sealed the plate with film, vortexed it for 30 sec, let it sit for 5 min, centrifuge briefly, placed it on magnetic plate for 5 min, and removed the clear supernatant carefully.
c) Added 180 μl of newly prepared 75% ethanol to each well. Held the bottom of the magnetic plate, moved the 96-well plate horizontally to the adjacent slot of the magnetic plate and back for 3-5 times, let the 96-well plate sit for 5 sec until the liquid became clear, then removed supernatant.
d) Repeat step c).
e) Removed the 96-well plate from the magnetic plate, centrifuged briefly, and put it back on the magnetic plate. Used 100-μl pipette to remove the remaining ethanol, and open the cover to dry until the pellet of magnetic beads appeared cracked slightly.
f) Added 27 μl of TE to each well and sealed the plate with membrane. Vortexed the plate for 30 sec, let it sit for 5 min, and centrifuged briefly. Placed the 96-well plate on magnetic plate and let it sit until the liquid became clear. Transferred 25 μL of supernatant as purified products to the corresponding well of a new 96-well plate for each well.

8. Ligation of sequencing adaptors. The reagents used were T4 DNA ligase (Thermo Fisher Scientific, EL0011) and DNA screening magnetic beads (Wuxi Biomeag Biotechnology Co., LTD., BMSX). The operating procedure is as follows: In this embodiment, the sequencing adaptor refers to the sequencing primer, and the sequencing primer used is the sequencing primer of Ion Torrent sequencing platform manufactured by Thermo Fisher Scientific. In this technical scheme, the choice of sequencing primers is affected by the sequencing instruments and sequencing reagents used, which does not constitute a limitation of this technical scheme; The ligase used is not limited to T4 ligase, any enzymes or other technical methods that are capable of ligating two DNA fragments are acceptable; The use of T4 ligase shall not be limited to the manufacturer or the item number herein; the proportion of components in the system and the reaction conditions are merely suitable conditions for the circumstance, does not constitute a limitation of the technical scheme. In this technical scheme, the description of purification method is the same as previously.
a) Added reagents in turn into the 96-well PCR plate according to the following table, vortexed for 5 sec, centrifuged briefly, and incubated the plate on PCR instrument at 25° C. for 30 min.

| Component | Volume (μl) |
|---|---|
| Blunt end DNA | 25 |
| Nuclease free ddH₂O | 10 |
| 10X ligase buffer | 5 |
| 50% PEG4000 | 5 |
| DNA ligase | 1 |
| Adaptor P1 | 2 |
| Index sequence X | 2 |
| Total system | 50 |

b) The reaction products were added to a 96-well plate preloaded with 40 μl of magnetic beads in each well, sealed the plate with membrane, vortexed for 30 sec, let the plate sit for 5 min, and centrifuged briefly. Placed the 96-well plate on magnetic plate for 5 min, and then carefully removed the clear supernatant.
c) Added 180 μl of newly prepared 75% ethanol to each well. Held the bottom of the magnetic plate, moved the 96-well plate horizontally to the adjacent slot of the magnetic plate and back for 3-5 times, let the 96-well plate sit for 5 sec until the liquid became clear, then removed supernatant.
d) Repeat step c).
e) Removed the 96-well plate from the magnetic plate, centrifuged briefly, and put it back on the magnetic plate. Used 100-μl pipette to remove the remaining ethanol, and open the cover to dry until the pellet of magnetic beads appeared cracked slightly.
f) Added 23 μl of TE to each well and sealed the plate with membrane. Vortexed the plate for 30 sec, let it sit for 5 min, and centrifuged briefly. Placed the 96-well plate on magnetic plate and let it sit until the liquid became clear. Transferred 20 μl of supernatant as purified products to the corresponding well of a new 96-well plate for each well.

9. Enrichment of sequencing library. The reagents used were HiFi high-fidelity Taq enzyme (KAPA biosystems, KK2602) and DNA screening magnetic beads (Wuxi Biomeag Biotechnology Co., LTD., BMSX). The operating procedure is as follows: In this embodiment, high-fidelity Taq enzyme is used to enrich sequencing libraries by PCR. In this technical scheme, enrichment of library is not essential. Omission of this step does not constitute an innovation of this technical scheme; Enrichment method is not limited to polymerase chain reaction (PCR), any method that is able to increase the proportion or amount of available sequencing library in the samples are acceptable; The enzyme used in PCR method is not limited to Taq polymerase, any other enzyme that is capable of amplifying nucleic acids is acceptable; Taq enzyme used is not limited to the manufacturer or the item number herein; The choice of PCR amplification primers is affected by the sequencing instruments and sequencing reagents used, and does not constitute a limitation of this technical scheme; the proportion of components in the system and the reaction conditions are merely suitable conditions for the circumstance, does not constitute a limitation of the technical scheme. In this technical scheme, the description of purification method is the same as before.
a) Prepared PCR reactions in a 96-well PCR plate according to the following table:

| Component | Reaction Volume (μl) |
|---|---|
| DNA | 20 |
| KAPA HiFi high fidelity enzyme mixture | 25 |
| PGM-A (10 μM) | 2.5 |
| PGM-P1 (10 μM) | 2.5 |
| Reaction total system | 50 |

### Reaction Condition:

| | Temperature | | Time | |
|---|---|---|---|---|
| 72° C. | | | | |
| 95° C. | | | 5 | min |
| | 15 cycles | 98° C. | 15 | sec |
| | | 62° C. | 30 | sec |
| | | 72° C. | 1 | min |

| | | |
|---|---|---|
| 72° C. | 2 | min |

| | |
|---|---|
| 4° C. | hold |

b) The reaction products were added to the corresponding wells of a 96-well plate preloaded with 40 μl magnetic beads, sealed the plate with membrane, vortexed for 30 sec, let sit for 5 min, and centrifuged briefly. Placed the plate on magnetic plate for 5 min and carefully removed the clear supernatant.
c) Added 180 μl of newly prepared 75% ethanol to each well. Held the bottom of the magnetic plate, moved the 96-well plate horizontally to the adjacent slot of the magnetic plate and back for 3-5 times, let the 96-well plate sit for 5 sec until the liquid became clear, then removed supernatant.
d) Repeat step c).
e) Removed the 96-well plate from the magnetic plate, centrifuged briefly, and put it back on the magnetic plate. Used 100-μl pipette to remove the remaining ethanol, and open the cover to dry until the pellet of magnetic beads appeared cracked slightly.
f) Added 52 μl of TE to each well and sealed the plate with membrane. Vortexed the plate for 30 sec, let it sit for 5 min, and centrifuged briefly. Placed the 96-well plate on magnetic plate and let it sit until the liquid became clear. Transferred 50 μl of supernatant to the corresponding well of a new 96-well plate for each well to perform additional purification following steps c) to f). Finally, 40 μl of TE was added for elute the final sequencing library from the magnetic beads.

10. Quantification of sequencing library. Qubit dsDNA BR Assay Kit 3.0 (Thermo Fisher Scientific, Q32850) was used to quantify the purified PCR products. Followed the instructions for reagents and instruments in the experiment. In this embodiment, sequencing library is quantified using a fluorescent dye method. In this technical scheme, quantification of a completed sequencing library is not limited to fluorescent dye method, may also be other dye method or non-dye method such as ultraviolet spectrophotometric method, fluorescent quantitative PCR method, capillary electrophoresis or microfluidic electrophoresis combined with nucleic acid dye fluorescence imaging methods; The fluorescent dye method is not limited to the manufacturer or the item number of the reagents herein; The method used is acceptable as long as it can accurately measure the mass of nucleic acids in the sample (refers to the amount of substance instead of quality). If ultraviolet spectrophotometry is used, nucleic acid quality information can be obtained, which also belongs to the scope of quality control in this technical scheme, and does not constitute an innovation of this technical scheme.

11. Pooling sequencing libraries. Sequencing libraries of different samples from the same batch were pooled together. Based on Qubit quantification results, equal amounts of libraries of different samples were added to make a mixed library. In this embodiment, sequencing libraries of different samples are mixed in equal amount. In this technical scheme, pooling libraries of different samples can also be unequal; the number of samples pooled each time can be adjusted flexibly according to the sequencing instruments, sequencing reagents, sequencing methods, actual experimental requirements and other conditions, which does not constitute a limitation of this technical scheme.

12. Quantification of pooled library. Used Ion Library TaqMan Quantitation Kit (Thermo Fisher Scientific, 4468802) and followed the reagent instructions. In this embodiment, the pooled library was quantified using fluorescent quantitative PCR. In this technical scheme, the quantitative method can also be other dye method or non-dye method, such as Qubit, ultraviolet spectrophotometry, Agilent 2100 biological analyzer, etc.; Fluorescent quantitative PCR method is not limited to the reagent of the manufacturer or the item number herein; Furthermore, this quantification step is not essential, and omission of this step does not constitute a limitation of this technical scheme; If ultraviolet spectrophotometry or Agilent 2100 and other instruments are used, nucleic acid quality information can be obtained, which also belongs to the scope of quality control of this technical scheme, and does not constitute an innovation of this technical scheme.

13. Sequencing. Using Ion One Touch 2 sequencing preparation platform, Ion Torrent PGM sequencing platform, and reagents including Ion PGM Hi-Q View OT2 Kit (Thermo Fisher Scientific, A29900), Ion PGM Hi-Q View Sequencing Kit (Thermo Fisher Scientific, A30044), and Ion 316 Chip Kit v2 BC (Thermo Fisher Scientific, 4488149), followed the instructions for the instruments and reagents to perform sequencing. In this embodiment, Ion Torrent PGM sequencer and Ion316 sequencing chip manufactured by Thermo Fisher Scientific as well as the supporting sequencing reagents and sequencing method were selected to conduct experiments. In this technical scheme, the NGS sequencer manufacturers compatible with the experimental process include but are not limited to Thermo Fisher Scientific, Illumina, BGI and other mainstream manufacturers as well as their instruments and reagents currently sold on the market and their sequencing methods, which do not constitute limitations of this technical scheme.

### 3. Data Analysis

Sequencing data are filtered to remove low-quality reads. The remaining high-quality clean data can be used for later analysis. The analysis process is as follows:

1. Database: Comprised 252 pathogenic microorganisms of clinical importance including 2,396 representative sequences. Method of selecting representative sequences was similar to the method used to select first tier seed in the aforementioned database construction process. The difference is that all sequences were clustered directly at 99.5% sequence similarity level in here and the seed of each cluster were selected to form a representative set.

2. The high-quality sequencing reads were aligned with hg19 version of human genome sequence using Bowtie2 with default parameters. Removed reads aligned to the human genome sequence.

3. Performed alignment between reads and sequences of representative set without competition among sequences for reads, and calculate read coverage for the sequences. The main methods were as follows: used Bowtie2 (parameter: −a; Or align reads with each reference sequence separately using default parameter) to perform alignment, then filtered alignments with 0.5% mismatch reads rate, and removed reads due to PCR duplication from alignments. The coverage of each reference sequence was calculated and filtered according to the parameters CV<=0.5 & gap<=15 & mean depth>=20.

4. Clustered the reference sequences resulted from filtering in step 3 according to 98% sequence similarity, and selected representative sequences of each cluster for further analysis in next step.

5. Aligned reads with the reference sequences obtained in step 4 using Bowtie2 software with default parameter, then filtered the alignments according to 0.5% mismatch reads rate, and removed reads due to PCR duplication from the alignments. Calculated CV, gap, Cor, CV/Cor, and mean depth for each reference sequence, and conducted iterative filtering of reference sequences with the requirement CV<=0.4 & Cor>=0.7 & mean depth>=20 & gap<=15 until all remaining reference sequences meet the requirement. The number and proportion of reads for each species were then calculated.

### 4. Experimental Results

The average number of sequencing reads of 10 samples was 55,931. Staphylococcus epidermidis was identified correctly in all samples with a sensitivity of 100%.

| | | | | Reference | Length of | | |
|---|---|---|---|---|---|---|---|
| | Total | | Valid | sequence | sequence | | Sequencing |
| | read | Identification | read | length | coverage | Sequencing | depth |
| Sample ID | count | result | count | (average) | (average) | coverage | (average) |
| INQ19M0101 | 59321 | Staphylococcus | 1884 | 1460 | 1460 | 1 | 376.49 |
| | | epidermidis | | | | | |
| INQ19M0102 | 58051 | Staphylococcus | 1835 | 1460 | 1460 | 1 | 381.10 |
| | | epidermidis | | | | | |
| INQ19M0103 | 57841 | Staphylococcus | 1868 | 1460 | 1460 | 1 | 428.19 |
| | | epidermidis | | | | | |
| INQ19M0104 | 59476 | Staphylococcus | 1794 | 1460 | 1460 | 1 | 402.32 |
| | | epidermidis | | | | | |
| INQ19M0105 | 57330 | Staphylococcus | 1762 | 1460 | 1460 | 1 | 363.91 |
| | | epidermidis | | | | | |
| INQ19M0106 | 37465 | Staphylococcus | 1517 | 1460 | 1460 | 1 | 323.54 |
| | | epidermidis | | | | | |
| INQ19M0107 | 55959 | Staphylococcus | 3015 | 1443 | 1441 | 0.998 | 297.56 |
| | | epidermidis | | | | | |
| INQ19M0108 | 55973 | Staphylococcus | 1220 | 1460 | 1460 | 1 | 239.12 |
| | | epidermidis | | | | | |
| INQ19M0109 | 54008 | Staphylococcus | 1220 | 1460 | 1460 | 1 | 214.30 |
| | | epidermidis | | | | | |
| INQ19M01010 | 63887 | Staphylococcus | 2701 | 1466.5 | 1466.5 | 1 | 214.30 |
| | | epidermidis | | | | | |

### Example 4

In this embodiment, Gram-negative bacteria were used as experimental subjects, which were a pure strain of known species cultured in liquid medium. The purpose is to investigate whether this technical scheme is capable of identifying correctly the microorganisms in the samples, and to investigate the limit of sensitivity in detection for this technical scheme.

### 1. Sample Preparation

Single species samples (Serratia marcescens) were cultured on agar plates and counts of colonies on the plates were used to confirm the number of bacterial CFU added to the samples. 2560 CFU of bacteria were added to each of samples INQ19M0111 and INQ19M0112; 640 CFU were added to INQ19M0113 and INQ19M0114; 160 CFU were added to INQ19M0115 and INQ19M0116; 40 CFU were added to INQ19M0117 and INQ19M0118. And 10 CFU were added to INQ19M0119 and INQ19M0120.

### 2. The Experiment Process

The same as example 3.

### 3. Data Analysis

The same as example 3.

### 4. Experimental Results

The average number of sequencing reads of 10 samples was 54,703. Except for sample INQ19M0119, Serratia marcescens was identified correctly in all samples.

| | | | | Reference | Length of | | |
|---|---|---|---|---|---|---|---|
| | Total | | Valid | sequence | sequence | | Sequencing |
| | read | Identification | read | length | coverage | Sequencing | depth |
| Sample ID | count | result | count | (average) | (average) | coverage | (average) |
| INQ19M01011 | 61892 | Serratia_marcescens | 6015 | 1409 | 1406 | 0.998 | 311.4 |
| INQ19M01012 | 55707 | Serratia_marcescens | 4844 | 1444 | 1440 | 0.997 | 303.8 |
| INQ19M01013 | 52507 | Serratia_marcescens | 8397 | 1416 | 1411 | 0.97 | 264. |
| INQ19M01014 | 58938 | Serratia_marcescens | 4853 | 1444 | 1440 | 0.97 | 299.0 |
| INQ19M01015 | 67275 | Serratia_marcescens | 6933 | 1432 | 1431 | 1.0 | 266.3 |
| INQ19M01016 | 66798 | Serratia_marcescens | 619 | 1453 | 1451 | 0.998 | 256.0 |
| INQ19M01017 | 59655 | Serratia_marcescens | 5318 | 1426 | 1425 | 0.999 | 249.0 |
| INQ19M01018 | 64886 | Serratia_marcescens | 689 | 1445 | 1440 | 0.996 | 236.8 |
| INQ19M01019 | 5828 | No significant | | | | | |
| | | pathogenic | | | | | |
| | | microorganism | | | | | |
| | | FOUND | | | | | |
| INQ19M01020 | 53540 | Serratia_marcescens | 2396 | 1379 | 1376 | 0.997 | 241.8 |

### Example 5

To investigate whether this technical scheme is able to identify microbial species correctly in mixed samples of multiple microbial species constituted from pure strains of known species cultured in liquid medium.

### 1. Sample Preparation

Five bacterial species (Staphylococcus aureus, Staphylococcus epidermidis, Serratia marcescens, Pseudomonas aeruginosa, Escherichia coli) were cultured on agar plates and colonies were counted to confirm the CFU numbers of bacteria. All strains were mixed equally in number (200 CFU) to generate samples INQ19M0133 and INQ19M0134.

### 2. Experimental Process

The same as example 3.

### 3. Data Analysis

The same as example 3.

### 4. Experimental Results

The average number of sequencing reads of 2 samples was 62,970, and all five species added in the samples were identified correctly.

| | | | | Reference | Length of | | |
|---|---|---|---|---|---|---|---|
| | Total | | Valid | sequence | sequence | | Sequencing |
| | read | Identification | read | length | coverage | Sequencing | depth |
| Sample ID | count | result | count | (average) | (average) | coverage | (average) |
| INQ19M0133 | 64099 | Pseudomonas | 1319 | 1345 | 1343 | 1 | 76 |
| | | aeruginosa | | | | | |
| | | Serratia | 2411 | 1464 | 1464 | 1 | 197.9 |
| | | marcescens | | | | | |
| | | Escherichia coli | 1270 | 1464 | 1463 | 1 | 210.4 |
| | | Staphylococcus | 2505 | 1432 | 1427 | 0.997 | 211 |
| | | aureus | | | | | |
| | | Staphylococcus | 1317 | 1460 | 1460 | 1 | 223.5 |
| | | epidermidis | | | | | |
| INQ19M0134 | 61841 | Pseudomonas | 1124 | 1353 | 1350 | 0.998 | 101.8 |
| | | aeruginosa | | | | | |
| | | Serratia | 3794 | 1444 | 1440 | 0.997 | 227.1 |
| | | marcescens | | | | | |
| | | Escherichia coli | 1141 | 1464 | 1462 | 1 | 203.9 |
| | | Staphylococcus | 2231 | 1386 | 1379.5 | 0.996 | 209.5 |
| | | aureus | | | | | |
| | | Staphylococcus | 1268 | 1460 | 1460 | 1 | 235.5 |
| | | epidermidis | | | | | |

### Example 6

The purpose of this embodiment is to investigate whether this technical scheme is able to identify correctly species of low proportion in samples containing multiple species using pure bacterial strains of known species cultured in liquid medium.

### 1. Sample Preparation

Two bacterial strains (Staphylococcus epidermidis and Serratia marcescens) were cultured on agar plates in laboratory and colonies on the plates were counted to confirm the CFU numbers of bacteria. Two samples, INQ19M0143 and INQ19M0144, were constituted by mixing the two bacterial strains in a 16-fold ratio in quantity (3200 CFU of Serratia marcescens and 200 CFU of Staphylococcus epidermidis).

### 2. Experiment Process

The same as example 3.

### 3. Data Analysis

The same as example 3.

### 4. Experimental Results

The average number of sequencing reads of 2 samples was 46,026. In both samples, the two species with 16 fold difference in quantity were identified correctly.

| | | | | Reference | Length of | | |
|---|---|---|---|---|---|---|---|
| | Total | | Valid | sequence | sequence | | Sequencing |
| | read | Identification | read | length | coverage | Sequencing | depth |
| Sample ID | count | result | count | (average) | (average) | coverage | (average) |
| INQ19M0143 | 59087 | Serratia | 5757 | 1432.25 | 1432.25 | 1 | 289.2 |
| | | marcescens | | | | | |
| | | Staphylococcus | 670 | 1450.5 | 1447 | 0.997 | 61.0 |
| | | epidermidis | | | | | |
| INQ19M0144 | 32244 | Serratia | 2315 | 1429.5 | 1424 | 0.996 | 202.5 |
| | | marcescens | | | | | |
| | | Staphylococcus | 393 | 1481 | 1480 | 0.999 | 67.6 |
| | | epidermidis | | | | | |

### Example 7

Investigated the ability of this technical scheme in clinical sample testing.

### 1. Sample Preparation

A bile specimen, INQ19M0322, was sampled in the hospital for identification of pathogenic microorganisms.

### 2. Experiment Process

The same as Example 3.

### 3. Data Analysis

1. Database: The database comprised 252 species of pathogenic microorganisms of clinical importance, including 1,920 first-tier representative sequences, with a total number of 143,009 sequences (redundancy in reference sequences was not removed from this version).

2. Aligned high-quality sequencing reads with hg19 version of the human genome sequence using Bowtie2 with default parameter. And reads aligned to the human genome sequence were removed.

3. Aligned sequencing reads to the first-tier seed reference sequences without competition for reads among reference sequences, calculated read coverage for reference sequences, and screened reference sequences. The main methods are: perform sequence alignment with Bowtie2 (parameter: −min-score L, −0.6, −0.15−a), the alignments are filtered at 0.5% mismatch reads rate, and reads in the alignments due to PCR duplication were removed. Then, calculate the read coverage for each reference sequence, and filter the alignments according to the threshold parameter CV<=0.5 & gap<=25 & mean depth>=20. The reference sequences passing the threshold are candidates for the next step.

4. Clustered the reference sequences passing through step 3 according to 98% sequence similarity and obtained seed representative sequence for each cluster to enter the next step of analysis.

5. Performed alignment wherein reference sequences competing for sequencing reads, and identified reference sequences that met the requirements through iterative screening based on read coverage. The specific steps are as follows: Aligned sequencing reads with the reference sequences output from step 4 using Bowtie2 software with default parameter, filtered the alignments according to 0.5% mismatch reads rate, and removed reads due to PCR duplication from the alignment. At base positions in the reference sequence alignment where the ratio between read count of the dominant base and that of the minor base was less than 0.95, kept only the reads whose base identity supports the reference sequence. Calculated CV, gap, Cor, CV/Cor and mean depth for each reference sequence, and then performed iterative screening using the threshold CV<=0.5 & Cor>=0.65 & mean depth>=0 & gap<=25 until all remaining reference sequences met the threshold requirements. Reference sequences that satisfy the requirements are candidates for the next step.

6. Performed iterative screening of the candidate seed sequences from step 5. The main steps are as follows: Firstly, classified these seeds according to their read coverage. The reference sequences meeting the threshold requirement CV<=0.4 & Cor>=0.9 & gap==0 entered directly into the final iterative screening; As to the reference sequences that did not meet the requirement, iterative screening was carried out for them respectively. The screening method was the same as step 3 and step 4, wherein the iterative screening parameter for the counterpart of step 3 were CV<=0.5 & gap<=15 & mean depth>=20 and the parameter for the counterpart of step 4 were CV<=0.5 & Cor>=0.7 & mean depth>=20 & gap<=25. All candidate seeds meeting the requirements were merged as the final candidate reference sequences.

7. Aligned reads with the final candidate reference sequences obtained in step 6, filtered out alignments with mismatch reads rate above 0.5%, and removed reads in the alignment due to PCR duplication. Calculated CV, gap, Cor, CV/Cor and mean depth for each reference sequence, and then performed iterative screening according to the threshold value CV<=0.5 & Cor>=0.7 & mean depth>=20 & gap<=25. All seeds that met the requirements were the final target sequences, and the corresponding species were the species detected. Meanwhile, the read count of each species and its proportion among all species in the sample were calculated.

### 4. Experimental Results

The total number of sequencing reads of the sample was 55,115, and 0.020% of all reads were of human origin. The test result of this technology showed that the sample contained Escherichia coli and Enterococcus faecium, which was consistent with the results obtained by microbial culturing methods.

| | | | | Reference | Length of | | |
|---|---|---|---|---|---|---|---|
| | Total | | Valid | sequence | sequence | | Sequencing |
| | read | Identification | read | length | coverage | Sequencing | depth |
| Sample ID | count | result | count | (average) | (average) | coverage | (average) |
| INQ190322 | 55115 | Enterococcus | 7707 | 1417.8 | 1417 | 0.999 | 349.0 |
| | | faecium | | | | | |
| | | Escherichia | 2761 | 1448 | 1445.25 | 0.998 | 140.8 |
| | | coli | | | | | |

### Example 8

Investigated the ability of this technical scheme in clinical sample testing.

### 1. Sample Preparation

Pleural fluid specimen (INQ19M0324) was sampled in a hospital for identification of pathogenic microorganisms.

### 2. Experiment Process

The same as Example 3.

### 3. Data Analysis

The same as Example 7.

### 4. Experimental Results

The total number of sequencing reads of the sample was 67,797, and 9.5% of all reads were of human origin. The test results of this technique showed that the sample contained Escherichia coli, which was consistent with the results obtained by using microbial culturing and other methods.

| | | | | Reference | Length of | | |
|---|---|---|---|---|---|---|---|
| | Total | | Valid | sequence | sequence | | Sequencing |
| | read | Identification | read | length | coverage | Sequencing | depth |
| Sample ID | count | result | count | (average) | (average) | coverage | (average) |
| INQ190324 | 67797 | Escherichia | 3136 | 1345 | 1345 | 1 | 397.5 |
| | | coli | | | | | |

### Example 9

Compared the performance of this technical scheme with that of the currently most commonly used method for microbiological testing. Performance indicators compared included positive rate of test and the rate of agreement between the two methods in samples tested positive. Investigated the ability of this technical scheme in clinical sample testing.

### 1. Sample Preparation

Samples for pathogen identification were collected from the hospital in August 2019. A total of 89 valid samples were collected. Each sample collected was divided into two parts, which were tested by this technical scheme and bacterial culturing method respectively. The types of samples include: bile, pleural effusion, joint effusion, cerebrospinal fluid, urine, pus, pericardial effusion, drainage fluid, sputum, etc., covering main sample types of clinical microbiological test.

### 2. Experimental Process

The same as Example 3.

### 3. Data Analysis

1. Database: contained more than 18,000 species (including all known bacteria, mycoplasma, chlamydia, rickettsia, and spirochete). The hierarchical relationship of reference sequences was constructed according to 97%, 98% and 99% sequence similarity, including 30,816 first-tier representative sequences, with a total number of 154,392 sequences (Redundancy was removed in this version of reference sequence database).

2. Aligned high-quality sequencing reads with hg38 version of the human genome reference sequence using Bowtie2 with default parameter. And removed reads aligned to the human genome reference sequence

3. Aligned reads with reference sequences in the database using Hisat2 software with parameter —score-min L, −1, −0.08—no-spliced-alignment—no-templatelen-adjustment—secondary-k 100000—mm—no-softclip, then filtered out alignments with mismatch reads rate above 1%, and removed reads in the alignment due to PCR duplication. Counted aligned reads for each reference sequence, labeled the reference sequence with read count greater than 10 as “has read aligned” or “no read aligned” otherwise. According to the clustering hierarchy and based on enrichment analysis method, seed IDs significantly enriched with reads were identified according to a relatively loose enrichment p value of 0.1. Retrieved corresponding seeds as candidate reference sequences for the next step.

4. Sequencing reads were aligned with candidate reference sequences obtained in step 3 using Bowtie2 with parameter setting —min-score L,−1,−0.1−a, wherein reference sequences did not compete for reads. Then, the alignments were filtered according to 0.5% mismatch reads rate, and reads in the alignment produced by PCR duplication were removed. Read coverage for each reference sequence was calculated, and the reference sequence was filtered using parameter CV<=0.6 & gap<=50 & mean depth>=20. The reference sequences passing the screening threshold became candidate sequences for the next step.

5. Sequencing reads were aligned with candidate reference sequences obtained from screening in step 4 using Bowtie2 software with the same parameter setting as in step 4. Then the alignments were filtered using 0.5% mismatch reads rate, and reads in the alignments generated by PCR duplication were removed. At sites within a reference sequence alignment where the proportion of the dominant allele was lower than 0.95, only reads with allele consistent with the reference sequence were retained. Calculated CV, gap, Cor and mean depth for each reference sequence, and performed iterative screening of reference sequences according to the requirement of CV<=0.6 & Cor>=0.65 & mean depth>=20 & gap<=25, until all remaining reference sequences met the requirement.

6.) If the read coverage of the reference sequences obtained in step 5 met the criteria CV<=0.4 & Cor>=0.9 & gap==0, then they entered into the final iteration directly. Otherwise, iterative screening was performed inside the second-tier cluster of the reference sequences wherein reference sequences inside the cluster competed for reads. The steps of internal iterative screening inside the second-tier cluster are the same as steps 4 and 5, wherein the threshold parameter for screening was CV<=0.6 & Cor>=0.6 & mean depth>=20 & gap<=25.

7. All reference sequences obtained in step 6 that met the requirements were combined to carry out the final iterative screening. Alignment with sequencing reads was performed using Bowtie2 with the same parameter setting as in step 4. Mismatch reads rate of 0.5% was used to filter the alignments, and reads generated due to PCR duplication were removed from the alignments. The read coverage for each reference sequence was calculated, based on which reference sequences were screened iteratively using threshold CV<=0.6 & Cor>=0.7 & mean depth>=20 & gap<=25. The reference sequences that ultimately met the requirements were the final target sequences, and the corresponding annotated species was the target species. In addition, the total read count for each species and its relative proportion in the sample were calculated.

### 4. Experimental Results

The positive rate of all samples tested using this technical scheme is 98.9% (88/89). Among samples tested positive by the bacterial culturing identification method, the rate of agreement with this technical scheme is 90.6% (48/53).

| | Results of culturing | Comparison with | identification results of | Sequencing | Proportion of |
|---|---|---|---|---|---|
| Sample ID | method | culturing method | this technical scheme | depth (average) | species |
| INQ19M0505 | Corynebacterium | match | Corynebacterium | 371.3 | 0.946 |
| | kroppenstedtii | | kroppenstedtii | | |
| | | | Escherichia coli | 20.8 | 0.053 |
| INQ19M0506 | Escherichia coli, | match | Citrobacter braakii | 403.2 | 0.239 |
| | Enterococcus faecalis | | Citrobacter freundii | 506.9 | 0.301 |
| | | | Citrobacter murliniae | 193.9331 | 0.115 |
| | | | Citrobacter young ae | 172.2 | 0.102 |
| | | | Enterococcus faecalis | 30.2 | 0.018 |
| | | | Raoultella planticola | 374.7 | 0.222 |
| INQ19M0507 | Enterococcus faecium, | match | Enterococcus faecium | 729.4 | 0.269 |
| | Klebsiella pneumoniae | | Enterococcus lactis | 175.0 | 0.0646 |
| | | | Klebsiella michiganensis | 1051.8 | 0.3882 |
| | | | Klebsiella oxytoca | 753.1 | 0.278 |
| INQ19M0508 | Escherichia coli | match | Cronobacter sakazakii | 655.5 | 0.2985 |
| | | | Escherichia coli | 1540.545 | 0.7015 |
| INQ19M0509 | negative | Culturing | Enterococcus faecalis | 188.1456 | 0.0782 |
| | | negative | Escherichia coli | 1522.115 | 0.6323 |
| | | | Klebsiella pneumoniae | 316.1937 | 0.1313 |
| | | | Shigella sonnei | 181.5587 | 0.0754 |
| | | | Sphingomonas | 199.3551 | 0.0828 |
| | | | paucimobilis | | |
| INQ19M0511 | Klebsiella pneumoniae | match | Klebsiella pneumoniae | 1566.49 | 1 |
| INQ19M0512 | Klebsiella pneumoniae | match | Enterococcus faecalis | 1030.899 | 0.4457 |
| | | | Klebsiella pneumoniae | 1281.956 | 0.5543 |
| INQ19M0513 | negative | Culturing | Acinetobacter | 117.6491 | 0.0518 |
| | | negative | calcoaceticus | | |
| | | | Acinetobacter seifertii | 112.9759 | 0.0498 |
| | | | Bacillus korlensis | 26.7662 | 0.0118 |
| | | | Brevundimonas aurantiaca | 200.4514 | 0.0883 |
| | | | Caulobacter vibrioides | 321.8307 | 0.1418 |
| | | | Enterococcus faecalis | 42.0576 | 0.0185 |
| | | | Escherichia coli | 732.4966 | 0.3228 |
| | | | Klebsiella pneumoniae | 162.9282 | 0.0718 |
| | | | Raoultella planticola | 30.8912 | 0.0136 |
| | | | Sphingomonas | 487.7002 | 0.2149 |
| | | | paucimobilis | | |
| | | | Staphylococcus aureus | 33.7761 | 0.0149 |
| INQ19M0514 | negative | Culturing | Aeromonas caviae | 35.2926 | 0.017 |
| | | negative | Dialister pneumosintes | 138.0549 | 0.0666 |
| | | | Enterococcus faecium | 292.5279 | 0.1412 |
| | | | Klebsiella aerogenes | 65.9466 | 0.0318 |
| | | | Lactobacillus iners | 89.1239 | 0.043 |
| | | | Prevotella bivia | 26.6786 | 0.0129 |
| | | | Prevotella timonensis | 33.6369 | 0.0162 |
| | | | Sphingomonas | 113.1857 | 0.0546 |
| | | | paucimobilis | | |
| | | | Staphylococcus aureus | 165.043 | 0.0797 |
| | | | Stenotrophomonas | 41.6973 | 0.0201 |
| | | | maltophilia | | |
| | | | Streptococcus agalactiae | 726.6547 | 0.3508 |
| | | | Streptococcus constellatus | 343.6676 | 0.1659 |
| INQ19M0515 | Enterococcus avium, | match | Dialister pneumosintes | 228.4501 | 0.2777 |
| | Streptococcus | | Enterococcus avium | 94.0342 | 0.1143 |
| | constellatus | | Prevotella bivia | 63.9387 | 0.0777 |
| | | | Prevotella timonensis | 44.6616 | 0.0543 |
| | | | Streptococcus constellatus | 391.4827 | 0.4759 |
| INQ19M0516 | Escherichia coli | match | Cronobacter sakazakii | 255.412 | 0.1472 |
| | | | Escherichia coli | 1479.724 | 0.8528 |
| INQ19M0519 | negative | Culturing | Citrobacter freundii | 149.4672 | 0.1654 |
| | | negative | Klebsiella pneumoniae | 664.6852 | 0.7354 |
| | | | Peptostreptococcus | 29.4604 | 0.0326 |
| | | | anaerobius | | |
| | | | Streptococcus agalactiae | 60.1907 | 0.0666 |
| INQ19M0520 | negative | Culturing | Klebsiella variicola | 105.9944 | 0.0493 |
| | | negative | Staphylococcus aureus | 2042.246 | 0.9507 |
| INQ19M0541 | Acinetobacter baumannii, | match | Acinetobacter baumannii | 1582.331 | 0.4646 |
| | Enterococcus faecium | | Enterococcus faecium | 1439.32 | 0.4226 |
| | | | Enterococcus lactis | 384.2295 | 0.1128 |
| INQ19M0542 | Enterococcus faecium | match | Acinetobacter baumannii | 228.2736 | 0.1171 |
| | | | Enterococcus faecium | 775.8785 | 0.3979 |
| | | | Enterococcus lactis | 374.5834 | 0.1921 |
| | | | Staphylococcus | 571.3549 | 0.293 |
| | | | epidermidis | | |
| INQ19M0543 | Acinetobacter baumannii | match | Acinetobacter baumannii | 290.4669 | 0.1219 |
| | Enterococcus faecium | | Enterococcus faecium | 1238.529 | 0.5197 |
| | | | Enterococcus lactis | 462.9707 | 0.1943 |
| | | | Staphylococcus | 391.3585 | 0.1642 |
| | | | epidermidis | | |
| INQ19M0544 | Klebsiella pneumoniae | match | Citrobacter braakii | 410.0082 | 0.1736 |
| | Pseudomonas aeruginosa | | Citrobacter freundii | 860.1433 | 0.3641 |
| | | | Citrobacter portucalensis | 211.5615 | 0.0896 |
| | | | Pseudomonas aeruginosa | 845.3372 | 0.3579 |
| | | | Staphylococcus aureus | 35.204 | 0.0149 |
| INQ19M0545 | Staphylococcus aureus | match | Staphylococcus aureus | 2746.739 | 1 |
| INQ19M0546 | negative | Culturing | Aeromonas hydrophila | 102.9105 | 0.0904 |
| | | negative | Agrobacterium | 98.6181 | 0.0867 |
| | | | larrymoorei | | |
| | | | Comamonas aquatica | 190.1049 | 0.1671 |
| | | | Enhydrobacter aerosaccus | 135.9048 | 0.1194 |
| | | | Klebsiella pneumoniae | 101.9084 | 0.0896 |
| | | | Leucob acter aridicollis | 78.5405 | 0.069 |
| | | | Moraxella osloensis | 271.1418 | 0.2383 |
| | | | Pseudochrobactrum | 73.1599 | 0.0643 |
| | | | saccharolyticum | | |
| | | | Pseudomonas putida | 85.6746 | 0.0753 |
| INQ19M0547 | negative | Culturing | Acinetobacter johnsonii | 75.9751 | 0.0376 |
| | | negative | Aerococcus viridans | 56.8927 | 0.0282 |
| | | | Citrobacter freundii | 314.3782 | 0.1557 |
| | | | Klebsiella pneumoniae | 168.54 | 0.0835 |
| | | | Pseudomonas aeruginosa | 1054.518 | 0.5223 |
| | | | Staphylococcus aureus | 199.2397 | 0.0987 |
| | | | [Pseudomonas] geniculata | 149.4197 | 0.074 |
| INQ19M0548 | Klebsiella pneumoniae | match | Klebsiella pneumoniae | 2928.427 | 1 |
| INQ19M0549 | Enterococcus faecium | match | Citrobacter murliniae | 171.4256 | 0.0756 |
| | | | Enterococcus faecium | 1296.014 | 0.5712 |
| | | | Enterococcus lactis | 207.6101 | 0.0915 |
| | | | Klebsiella pneumoniae | 593.7455 | 0.2617 |
| INQ19M0550 | negative | Culturing | Paracoccus sphaerophysae | 32.9443 | 0.1195 |
| | | negative | Serratia marcescens | 121.6232 | 0.4411 |
| | | | Sphingomonas | 78.6106 | 0.2851 |
| | | | paucimobilis | | |
| | | | Stenotrophomonas | 42.5257 | 0.1542 |
| | | | maltophilia | | |
| INQ19M0551 | negative | Culturing | Acinetobacter johnsonii | 86.5267 | 0.0477 |
| | | negative | Citrobacter freundii | 257.7489 | 0.1419 |
| | | | Coryneb acterium | 220.3264 | 0.1213 |
| | | | tuberculostearicum | | |
| | | | Pseudomonas aeruginosa | 503.7701 | 0.2774 |
| | | | Sphingomonas abaci | 218.9324 | 0.1206 |
| | | | Sphingomonas | 23.1694 | 0.0128 |
| | | | paucimobilis | | |
| | | | Sphingomonas roseiflava | 113.5798 | 0.0626 |
| | | | Staphylococcus hominis | 61.072 | 0.0336 |
| | | | [Pseudomonas] geniculata | 330.695 | 0.1821 |
| INQ19M0552 | Stenotrophomonas | match | Staphylococcus | 1044.602 | 0.697 |
| | maltophilia | | haemolyticus | | |
| | | | Stenotrophomonas | 226.1568 | 0.1509 |
| | | | maltophilia | | |
| | | | [Pseudomonas] geniculata | 227.8832 | 0.1521 |
| INQ19M0553 | negative | match | NO significant pathogenic | | |
| | | | microorganism FOUND | | |
| INQ19M0554 | Stenotrophomonas | match | Stenotrophomonas | 1051.743 | 0.7377 |
| | maltophilia | | maltophilia | | |
| | | | [Pseudomonas] geniculata | 374.0404 | 0.2623 |
| INQ19M0555 | negative | Culturing | Acinetobacter | 348.4726 | 0.1437 |
| | | negative | calcoaceticus | | |
| | | | Aerococcus urinaeequi | 187.0825 | 0.0771 |
| | | | Aerococcus viridans | 81.7212 | 0.0337 |
| | | | Cutibacterium acnes | 49.3784 | 0.0204 |
| | | | Dietzia cercidiphylli | 87.2552 | 0.036 |
| | | | Dietzia dagingensis | 85.7062 | 0.0353 |
| | | | Dietzia natronolimnaea | 99.2994 | 0.0409 |
| | | | Exiguobacterium | 313.6466 | 0.1293 |
| | | | acetylicum | | |
| | | | Micrococcus flavus | 66.8485 | 0.0276 |
| | | | Pantoea brenneri | 82.9494 | 0.0342 |
| | | | Paracoccus sphaerophysae | 197.1446 | 0.0813 |
| | | | Pseudomonas aeruginosa | 206.4015 | 0.0851 |
| | | | Pseudomonas putida | 118.0001 | 0.0487 |
| | | | Sphingomonas | 162.394 | 0.067 |
| | | | paucimobilis | | |
| | | | Staphylococcus hominis | 91.3009 | 0.0376 |
| | | | Stenotrophomonas | 247.4061 | 0.102 |
| | | | maltophilia | | |
| INQ19M0556 | Enterococcus faecium | match | Enterococcus faecium | 1188.233 | 0.4813 |
| | Pseudomonas aeruginosa | | Enterococcus lactis | 594.6852 | 0.2409 |
| | | | Pseudomonas aeruginosa | 685.6458 | 0.2778 |
| INQ19M0557 | negative | Culturing | Lactobacillus iners | 222.0121 | 0.2483 |
| | | negative | Prevotella timonensis | 49.2954 | 0.0551 |
| | | | Pseudomonas mendocina | 103.3646 | 0.1156 |
| | | | Staphylococcus hominis | 94.074 | 0.1052 |
| | | | Stenotrophomonas | 312.3051 | 0.3493 |
| | | | maltophilia | | |
| | | | Streptococcus equinus | 113.0678 | 0.1265 |
| INQ19M0558 | Klebsiella pneumoniae | match | Citrobacter freundii | 657.5046 | 0.2731 |
| | | | Klebsiella pneumoniae | 1660.694 | 0.6897 |
| | | | Peptostreptococcus | 89.6838 | 0.0372 |
| | | | anaerobius | | |
| INQ19M0559 | negative | Culturing | Aeromonas hydrophila | 63.6679 | 0.0537 |
| | | negative | Brevundimonas aurantiaca | 136.9118 | 0.1155 |
| | | | Klebsiella pneumoniae | 65.2064 | 0.055 |
| | | | Mycoplasma hominis | 415.7812 | 0.3508 |
| | | | Sphingomonas | 288.3133 | 0.2432 |
| | | | paucimobilis | | |
| | | | Stenotrophomonas | 27.7378 | 0.0234 |
| | | | maltophilia | | |
| | | | Ureaplasma parvum | 187.7436 | 0.1584 |
| INQ19M0560 | Aeromonas hydrophila | match | Aeromonas hydrophila | 785.0683 | 0.5496 |
| | | | Klebsiella aerogenes | 147.3108 | 0.1031 |
| | | | Klebsiella pneumoniae | 241.8182 | 0.1693 |
| | | | Klebsiella variicola | 254.289 | 0.178 |
| INQ19M0561 | Escherichia coli | match | Escherichia coli | 1987.987 | 0.7735 |
| | | | Shigella boydii | 226.6346 | 0.0882 |
| | | | Streptococcus | 355.6084 | 0.1384 |
| | | | pasteurianus | | |
| INQ19M0562 | negative | Culturing | Escherichia coli | 126.2312 | 0.2234 |
| | | negative | Klebsiella variicola | 26.7045 | 0.0473 |
| | | | Mycoplasma hominis | 359.0215 | 0.6354 |
| | | | Ureaplasma parvum | 53.1112 | 0.094 |
| INQ19M0563 | Stenotrophomonas | match | Stenotrophomonas | 641.6114 | 1 |
| | maltophilia | | maltophilia | | |
| INQ19M0568 | Citrobacter freundii | match | Citrobacter freundii | 604.4618 | 0.5635 |
| | Klebsiella pneumoniae | | Klebsiella pneumoniae | 431.9256 | 0.4027 |
| | | | Peptostreptococcus | 36.2236 | 0.0338 |
| | | | anaerobius | | |
| INQ19M0569 | Morganella morganii | match | Morganella morganii | 1065.305 | 0.827 |
| | | | Ureaplasma urealyticum | 222.9003 | 0.173 |
| INQ19M0570 | Stenotrophomonas | match | Stenotrophomonas | 1015.226 | 0.8286 |
| | maltophili | | maltophilia | | |
| | | | [Pseudomonas] geniculata | 210.0028 | 0.1714 |
| INQ19M0571 | negative | Culturing | Brevundimonas aurantiaca | 323.8532 | 0.4372 |
| | | negative | Sphingomonas | 416.9497 | 0.5628 |
| | | | paucimobilis | | |
| INQ19M0572 | Klebsiella aerogenes | match | Klebsiella aerogenes | 1302.243 | 1 |
| INQ19M0574 | Enterococcus faecalis | match | Acinetobacter baumannii | 35.3957 | 0.0185 |
| | Klebsiella pneumoniae | | Enterococcus faecalis | 910.0068 | 0.476 |
| | | | Enterococcus faecium | 132.3774 | 0.0692 |
| | | | Escherichia coli | 37.0312 | 0.0194 |
| | | | Klebsiella pneumoniae | 796.8001 | 0.4168 |
| INQ19M0578 | negative | Culturing | Mycoplasma hominis | 412.1882 | 0.7834 |
| | | negative | Ureaplasma parvum | 113.991 | 0.2166 |
| INQ19M0579 | Corynebacterium gergeri | don't match | Morganella morganii | 1157.943 | 1 |
| INQ19M0580 | negative | Culturing | Citrobacter freundii | 137.2911 | 0.061 |
| | | negative | Clostridium perfiringens | 27.4796 | 0.0122 |
| | | | Enterococcus casseliflavus | 133.7001 | 0.0594 |
| | | | Enterococcus faecalis | 1297.182 | 0.5759 |
| | | | Escherichia coli | 471.0469 | 0.2091 |
| | | | Klebsiella oxytoca | 120.8912 | 0.0537 |
| | | | Paracoccus sphaerophysae | 21.7166 | 0.0096 |
| | | | Stenotrophomonas | 42.9611 | 0.0191 |
| | | | maltophilia | | |
| INQ19M0581 | Pseudomonas aeruginosa | match | Pseudomonas aeruginosa | 1850.078 | 1 |
| INQ19M0582 | negative | Culturing | Cedecea davisae | 119.2078 | 0.2044 |
| | | negative | Citrobacter fireundii | 197.2868 | 0.3384 |
| | | | Citrobacter portucalensis | 89.4932 | 0.1535 |
| | | | Pseudomonas stutzeri | 177.0853 | 0.3037 |
| INQ19M0583 | Enterococcus faecium | match | Enterococcus faecium | 1669.447 | 0.8334 |
| | | | Enterococcus lactis | 333.6598 | 0.1666 |
| INQ19M0584 | negative | Culturing | Pseudomonas aeruginosa | 1902.842 | 0.8463 |
| | | negative | Stenotrophomonas | 24.3442 | 0.0108 |
| | | | maltophilia | | |
| | | | Streptococcus anginosus | 321.3437 | 0.1429 |
| INQ19M0585 | Enterococcus faecalis, | match | Citrobacter freundii | 246.3088 | 0.0999 |
| | Pseudomonas Mendoza | | Clostridium perfiringens | 166.5411 | 0.0675 |
| | | | Clostridium sporogenes | 84.7727 | 0.0344 |
| | | | Enterococcus casseliflavus | 524.3258 | 0.2127 |
| | | | Enterococcus faecalis | 1443.623 | 0.5855 |
| INQ19M0586 | Enterococcus faecium | match | Enterococcus durans | 260.8094 | 0.1077 |
| | | | Enterococcus faecium | 1608.986 | 0.6647 |
| | | | Enterococcus lactis | 550.7194 | 0.2275 |
| INQ19M0587 | Escherichia coli | match | Bacteroides dorei | 274.2001 | 0.11 |
| | | | Bacteroides uniformis | 39.7384 | 0.0159 |
| | | | Cronobacter sakazakii | 189.8262 | 0.0761 |
| | | | Enterococcus faecium | 701.7919 | 0.2814 |
| | | | Enterococcus lactis | 123.8405 | 0.0497 |
| | | | Escherichia coli | 729.2225 | 0.2924 |
| | | | Morganella morganii | 173.1165 | 0.0694 |
| | | | Parabacteroides distasonis | 37.4789 | 0.015 |
| | | | Pseudomonas sihuiensis | 224.4366 | 0.09 |
| INQ19M0588 | negative | Culturing | Enterococcus faecalis | 57.6644 | 1 |
| | | negative | | | |
| INQ19M0589 | negative | Culturing | Brevibacillus brevis | 160.7148 | 0.088 |
| | | negative | Brevibacillus formosus | 77.514 | 0.0424 |
| | | | Citrobacter freundii | 162.3993 | 0.0889 |
| | | | Citrobacter portucalensis | 76.4829 | 0.0419 |
| | | | Comamonas testosteroni | 159.8642 | 0.0875 |
| | | | Comamonas thiooxydans | 79.6648 | 0.0436 |
| | | | Cutibacterium acnes | 93.0873 | 0.051 |
| | | | Dietzia cercidiphylli | 148.9152 | 0.0815 |
| | | | Empedobacter falsenii | 353.1308 | 0.1934 |
| | | | Ralstonia pickettii | 183.8418 | 0.1007 |
| | | | Sphingomonas | 92.0234 | 0.0504 |
| | | | paucimobilis | | |
| | | | Sphingomonas rhizogenes | 108.3933 | 0.0593 |
| | | | Staphylococcus wameri | 130.3169 | 0.0714 |
| INQ19M0590 | Klebsiella pneumoniae | match | Klebsiella pneumoniae | 2191.677 | 1 |
| INQ19M0591 | Enterococcus faecium | match | Enterococcus faecium | 1222.75 | 0.7558 |
| | | | Enterococcus lactis | 395.1259 | 0.2442 |
| INQ19M0592 | negative | Culturing | Citrobacter freundii | 30.5895 | 1 |
| | | negative | | | |
| INQ19M0593 | negative | Culturing | Aerococcus urinaeequi | 120.7224 | 0.2571 |
| | | negative | Aeromonas caviae | 33.4288 | 0.0712 |
| | | | Citrobacter freundii | 56.7367 | 0.1208 |
| | | | Cutibacterium acnes | 36.389 | 0.0775 |
| | | | Enterob acter hormaechei | 50.752 | 0.1081 |
| | | | Leuconostoc lactis | 41.0523 | 0.0874 |
| | | | Pseudomonas koreensis | 66.1288 | 0.1408 |
| | | | Staphylococcus hominis | 27.2322 | 0.058 |
| | | | Stenotrophomonas | 37.1503 | 0.0791 |
| | | | maltophilia | | |
| INQ19M0594 | Citrobacter freundii | match | Citrobacter freundii | 286.9758 | 0.7664 |
| | Stenotrophomonas | | Stenotrophomonas | 87.4817 | 0.2336 |
| | maltophilia | | maltophilia | | |
| INQ19M0595 | Klebsiella aerogenes | match | Acinetobacter soli | 37.9767 | 0.0226 |
| | | | Klebsiella aerogenes | 1580.69 | 0.9424 |
| | | | Stenotrophomonas | 58.6841 | 0.035 |
| | | | acidaminiphila | | |
| INQ19M0596 | Acinetob acter baumannii | match | Acinetob acter baumannii | 608.1492 | 0.2745 |
| | Enterococcus faecium | | Enterococcus faecium | 1291.509 | 0.583 |
| | | | Enterococcus lactis | 315.5438 | 0.1424 |
| INQ19M0597 | negative | Culturing | Acinetob acter indicus | 700.2803 | 0.345 |
| | | negative | Brooklawnia massiliensis | 41.706 | 0.0205 |
| | | | Citrobacter braakii | 236.2254 | 0.1164 |
| | | | Comamonas testosteroni | 74.0236 | 0.0365 |
| | | | Coryneb acterium | 25.109 | 0.0124 |
| | | | tuberculostearicum | | |
| | | | Enterob acter cloacae | 109.472 | 0.0539 |
| | | | Enterob acter hormaechei | 232.4755 | 0.1145 |
| | | | Enterococcus faecalis | 44.1421 | 0.0217 |
| | | | Klebsiella pneumoniae | 237.9309 | 0.1172 |
| | | | Kocuria palustris | 82.3203 | 0.0406 |
| | | | Pseudomonas monteilii | 76.9349 | 0.0379 |
| | | | Staphylococcus hominis | 63.8688 | 0.0315 |
| | | | Stenotrophomonas | 105.5359 | 0.052 |
| | | | maltophilia | | |
| INQ19M0600 | negative | Culturing | Staphylococcus cohnii | 67.8082 | 1 |
| | | negative | | | |
| INQ19M0601 | negative | Culturing | Acinetob acter | 421.7884 | 0.5067 |
| | | negative | nosocomialis | | |
| | | | Brevundimonas aurantiaca | 249.7288 | 0.3 |
| | | | Sphingomonas | 160.9718 | 0.1934 |
| | | | paucimobilis | | |
| INQ19M0602 | Klebsiella aerogenes | match | Acinetob acter soli | 22.3956 | 0.0177 |
| | | | Klebsiella aerogenes | 1245.9 | 0.9823 |
| INQ19M0603 | Pseudomonas aeruginosa | match | Enterococcus faecalis | 613.5863 | 0.2718 |
| | | | Lactobacillus rhamnosus | 48.1325 | 0.0213 |
| | | | Pseudomonas aeruginosa | 1595.747 | 0.7069 |
| INQ19M0604 | Escherichia coli | match | Cronob acter sakazakii | 410.5023 | 0.1359 |
| | | | Escherichia coli | 2460.951 | 0.8149 |
| | | | Shigella sonnei | 148.6016 | 0.0492 |
| INQ19M0605 | Enterococcus faecium | match | Enterococcus faecium | 1466.529 | 0.7098 |
| | | | Enterococcus lactis | 572.0925 | 0.2769 |
| | | | Escherichia coli | 27.4105 | 0.0133 |
| INQ19M0606 | Enterococcus faecium | match | Enterococcus faecium | 1108.599 | 0.5271 |
| | | | Enterococcus lactis | 534.4012 | 0.2541 |
| | | | Escherichia coli | 460.3777 | 0.2189 |
| INQ19M0607 | Staphylococcus aureus | match | Acinetob acter junii | 1380.241 | 0.5645 |
| | Enterococcus faecium | | Staphylococcus pasteuri | 225.2554 | 0.0921 |
| | | | Staphylococcus wameri | 450.1579 | 0.1841 |
| | | | Streptococcus sanguinis | 389.3095 | 0.1592 |
| INQ19M0608 | negative | Culturing | Dietzia cercidiphylli | 56.7756 | 0.0223 |
| | | negative | Enterococcus faecium | 214.3476 | 0.0842 |
| | | | Paracoccus marcusii | 123.2046 | 0.0484 |
| | | | Pseudomonas stutzeri | 97.4395 | 0.0383 |
| | | | Pseudomonas | 81.4121 | 0.032 |
| | | | toyotomiensis | | |
| | | | Sphingomonas | 83.5783 | 0.0328 |
| | | | paucimobilis | | |
| | | | Staphylococcus aureus | 1611.484 | 0.6332 |
| | | | Stenotrophomonas | 276.8388 | 0.1088 |
| | | | maltophilia | | |
| INQ19M0609 | Enterococcus faecalis | match | Enterococcus faecalis | 1935.744 | 0.9495 |
| | | | Streptococcus mitis | 80.8991 | 0.0397 |
| | | | Streptococcus vestibularis | 22.0721 | 0.0108 |
| INQ19M0610 | Klebsiella | match | Klebsiella pneumoniae | 1917.544 | 0.7567 |
| | pneumoniae | | Stenotrophomonas | 616.5734 | 0.2433 |
| | | | maltophilia | | |
| INQ19M0611 | negative | Culturing | Acinetobacter | 99.7061 | 0.1454 |
| | | negative | nosocomialis | | |
| | | | Bacillus korlensis | 51.7749 | 0.0755 |
| | | | Brevundimonas aurantiaca | 27.3259 | 0.0398 |
| | | | Sphingomonas | 506.9203 | 0.7392 |
| | | | paucimobilis | | |
| INQ19M0612 | Staphylococcus aureus, | match | Enterococcus faecium | 711.8751 | 0.4283 |
| | Staphylococcus vohs, | | Enterococcus lactis | 119.499 | 0.0719 |
| | | | Staphylococcus aureus | 830.7888 | 0.4998 |
| INQ19M0613 | negative | Culturing | Bacillus nealsonii | 104.8224 | 0.1614 |
| | | negative | Enterob acter hormaechei | 466.4263 | 0.7181 |
| | | | Lactobacillus fermentum | 78.3103 | 0.1206 |
| INQ19M0614 | negative | Culturing | Cutibacterium acnes | 230.7726 | 0.0965 |
| | | negative | Dietzia cercidiphylli | 176.8789 | 0.074 |
| | | | Klebsiella pneumoniae | 481.6676 | 0.2015 |
| | | | Pseudomonas mendocina | 208.1528 | 0.0871 |
| | | | Pseudomonas | 394.927 | 0.1652 |
| | | | toyotomiensis | | |
| | | | Staphylococcus hominis | 367.8681 | 0.1539 |
| | | | Streptococcus | 530.2568 | 0.2218 |
| | | | thermophilus | | |
| INQ19M0615 | Gardnerella vaginalis | don't match | NO significant pathogenic | | |
| | | | microorganism FOUND | | |
| INQ19M0616 | negative | Culturing | Acinetobacter | 24.5116 | 0.0448 |
| | | negative | calcoaceticus | | |
| | | | Brevundimonas aurantiaca | 79.2781 | 0.145 |
| | | | Dietzia natronolimnaea | 55.4863 | 0.1015 |
| | | | Pseudomonas monteilii | 38.4283 | 0.0703 |
| | | | Pseudomonas stutzeri | 132.4651 | 0.2422 |
| | | | Rhizobium sphaerophysae | 72.0626 | 0.1318 |
| | | | Sphingomonas | 144.601 | 0.2644 |
| | | | paucimobilis | | |
| INQ19M0617 | Klebsiella pneumoniae | match | Klebsiella pneumoniae | 3050.95 | 1 |
| INQ19M0618 | Acid-producing Klebsiella | don't match | NO significant pathogenic | | |
| | | | microorganism FOUND | | |
| INQ19M0619 | Proteus mirabilis | don't match | Finegoldia magna | 43.5983 | 0.0306 |
| | | | Peptostreptococcus | 370.5638 | 0.2599 |
| | | | stomatis | | |
| | | | Proteus hauseri | 161.7394 | 0.1134 |
| | | | Proteus vulgaris | 751.417 | 0.527 |
| | | | Solobacterium moorei | 74.4195 | 0.0522 |
| | | | Streptococcus anginosus | 24.2294 | 0.017 |
| INQ19M0620 | negative | Culturing | Dietzia cercidiphylli | 112.4837 | 0.0845 |
| | | negative | Dietzia natronolimnaea | 119.2449 | 0.0896 |
| | | | Finegoldia magna | 277.1295 | 0.2082 |
| | | | Peptostreptococcus | 380.9791 | 0.2863 |
| | | | stomatis | | |
| | | | Proteus penneri | 179.0938 | 0.1346 |
| | | | Solobacterium moorei | 261.895 | 0.1968 |
| INQ19M0621 | negative | Culturing | Enterococcus faecium | 155.2136 | 0.0895 |
| | | negative | Fusobacterium nucleatum | 21.6664 | 0.0125 |
| | | | Morganella morganii | 129.8331 | 0.0749 |
| | | | Peptostreptococcus | 343.0613 | 0.1979 |
| | | | stomatis | | |
| | | | Proteus hauseri | 147.8853 | 0.0853 |
| | | | Proteus vulgaris | 711.8955 | 0.4107 |
| | | | Solobacterium moorei | 120.3407 | 0.0694 |
| | | | Streptococcus anginosus | 81.2966 | 0.0469 |
| | | | [Ruminococcus] gnavus | 22.1923 | 0.0128 |
| INQ19M0622 | Klebsiella pneumonia | match | Klebsiella pneumoniae | 2934.712 | 1 |
| INQ19M0624 | Enterob acter cloacae | match | Enterob acter asburiae | 333.8736 | 0.1818 |
| | Enterococcus faecium | | Enterob acter cloacae | 169.8272 | 0.0925 |
| | | | Enterob acter ludwigii | 164.857 | 0.0898 |
| | | | Enterob acter | 192.4536 | 0.1048 |
| | | | roggenkampii | | |
| | | | Enterococcus durans | 240.7372 | 0.1311 |
| | | | Enterococcus faecium | 493.0004 | 0.2685 |
| | | | Enterococcus lactis | 241.3386 | 0.1314 |
| INQ19M0625 | negative | Culturing | Acinetob acter | 181.8795 | 0.1613 |
| | | negative | nosocomialis | | |
| | | | Bacillus korlensis | 20.874 | 0.0185 |
| | | | Enterococcus faecium | 41.1439 | 0.0365 |
| | | | Peptostreptococcus | 203.1406 | 0.1802 |
| | | | stomatis | | |
| | | | Proteus hauseri | 86.005 | 0.0763 |
| | | | Proteus vulgaris | 164.4031 | 0.1458 |
| | | | Solobacterium moorei | 73.4257 | 0.0651 |
| | | | Sphingomonas | 307.644 | 0.2729 |
| | | | paucimobilis | | |
| | | | Streptococcus anginosus | 48.8106 | 0.0433 |
| INQ19M0626 | negative | Culturing | Dietzia cercidiphylli | 95.9993 | 0.1292 |
| | | negative | Dietzia natronolimnaea | 107.0021 | 0.144 |
| | | | Enterococcus faecium | 508.5943 | 0.6844 |
| | | | Pseudomonas stutzeri | 31.5404 | 0.0424 |
| INQ19M0627 | negative | Culturing | Acinetob acter | 216.3945 | 0.0943 |
| | | negative | nosocomialis | | |
| | | | Bacillus korlensis | 40.934 | 0.0178 |
| | | | Brevundimonas aurantiaca | 21.6785 | 0.0095 |
| | | | Clostridium perfringens | 28.1317 | 0.0123 |
| | | | Enterococcus faecium | 53.3983 | 0.0233 |
| | | | Enterococcus lactis | 51.3662 | 0.0224 |
| | | | Klebsiella pneumoniae | 130.4254 | 0.0569 |
| | | | Morganella morganii | 115.4799 | 0.0503 |
| | | | Peptostreptococcus | 323.2473 | 0.1409 |
| | | | stomatis | | |
| | | | Proteus hauseri | 134.0248 | 0.0584 |
| | | | Proteus vulgaris | 663.5412 | 0.2893 |
| | | | Solobacterium moorei | 106.7602 | 0.0465 |
| | | | Sphingomonas | 309.6485 | 0.135 |
| | | | paucimobilis | | |
| | | | Streptococcus anginosus | 64.7386 | 0.0282 |
| | | | [Ruminococcus] gnavus | 34.0007 | 0.0148 |

### Example 10

This embodiment illustrated that, in this technical scheme, the addition of a certain amount of nucleic acids of known sequence at a certain step of the experimental process can improve the ability to identify potential contamination results in test results.

### 1. Sample Preparation

Drainage fluid specimens were collected from the hospital to test for pathogenic microorganisms. Each sample collected was divided into two parts, which were tested by this technical scheme and bacterial culturing method respectively.

### 2. Experiment Process

The process is basically the same as Example 3. The added operations include: Step 2, When the target gene was amplified, a certain amount of plasmid containing nucleic acids of a known sequence is added into the PCR reaction solution. The nucleic acid extraction products obtained from the negative control during sample extraction were also added with the same amount of nucleic acids of a known sequence. All reactions were carried out together. The sequence length of the added nucleic acids was similar to that of the 16S rRNA gene. This embodiment chose to add the nucleic acids of a known sequence at this step. In this technical scheme, the addition of the nucleic acids of a known sequence is not essential. Failure to do so does not affect the testing capability of this technical scheme; Addition of the nucleic acids of a known sequence is not limited to this step, it may also occur before nucleic acid extraction, or after this step, or at any step considered appropriate in this technical scheme; The added nucleic acid sequence can exist alone, or together with carriers such as plasmid, virus, cell, etc.; The number of nucleic acid sequences added can range from one to unlimited copies.

### 3. Data Analysis

1. Database: contained 2,202 clinically pathogenic bacterial species. The hierarchical relationship of reference sequences was constructed according to 97%, 98% and 99% sequence similarity, including 40,035 first-tier representative sequences, with a total number of 92,385 sequences (Redundancy was removed in this reference sequence database).

2. Aligned reads with reference sequences in the database using Hisat2 software with parameter —score-min L,−1,−0.08—no-spliced-alignment—no-templatelen-adjustment—secondary-k 100000—mm—no-softclip, then filtered out alignments with mismatch reads rate above 1%, and removed reads in the alignment due to PCR duplication. Counted aligned reads for each reference sequence, labeled the reference sequence with read count greater than 10 as “has read aligned” or “no read aligned” otherwise. According to the clustering hierarchy and based on enrichment analysis method, seed IDs enriched significantly with reads were identified according to a relatively loose enrichment p value of 0.1. Retrieved corresponding seeds as candidate reference sequences for the next step.

3. Sequencing reads were aligned with candidate reference sequences obtained in step2 using Bowtie2 with parameter setting—min-score L,−1,−0.08−a, wherein reference sequences did not compete for reads. Then, the alignments were filtered according to 1% mismatch reads rate, and reads in the alignment produced by PCR duplication were removed. Read coverage for each reference sequence was calculated, and the reference sequence was filtered using parameter CV<=0.6 & end gap<=40 & middle gap<=10 & mean depth>=20. The reference sequences passing the screening threshold became candidate sequences for the next step.

4. The reference sequences obtained in step 3 was randomly grouped by means of permutation and combination, and then performed alignment and iterative screening in each group. Reads were aligned with reference sequences of each group separately using Bowtie2 software with the same parameter as that in step 3. The alignments were filtered according to 1% mismatch reads rate, and reads in alignments produced by PCR duplication were removed. NRMSE, end gap, middle gap, Cor, mean depth and other metrics for each reference sequence were calculated. Reference sequences were screened iteratively according to the requirement of NRMSE<=0.35 & Cor<=0.6 & mean depth>=20 & end gap<=40 & middle gap<=10, until all remaining reference sequences met the requirement.

5. Competitive iterative screening was carried out inside the second-tier clusters separately of all the reference sequences obtained in step 4. The processes of iterative screening inside second-tier clusters were the same as those of step 3 and 4, but the filtering threshold was more stringent: the alignment parameter was—min-score L,−1,−0.04−a; Only 0.5% mismatch reads rate was allowed in both steps; In the step equivalent to step 3, the filtering parameter was end gap<=25 & middle gap<1 & CV<=0.55 & mean depth>=20; In the step equivalent to step 4, the filtering parameter was NRMSE<=0.3 & Cor>=0.6 & mean depth>=20 & end gap<=25 & middle gap<1. The reference sequences that met the requirements entered the next step of analysis.

6. All reference sequences obtained in step 5 that met the requirements were combined to carry out the final iterative screening. Alignment with sequencing reads was performed using Bowtie2 with the same parameter setting as in step 5. Mismatch reads rate of 0.5% was used to filter the alignments, and reads generated due to PCR duplication were removed from the alignments. At sites within a reference sequence alignment where the proportion of the dominant allele was lower than 0.95, only reads with allele consistent with the reference sequence were retained. The read coverage for each reference sequence was calculated, and reference sequences were screened iteratively using threshold NRMSE<=0.3 & Cor>=0.65 & mean depth>=20 & end gap<=25 & middle gap<1. The reference sequences that ultimately met the requirements were the final target sequences, and the corresponding annotated species was the target species. In addition, the total read count for each species and its relative proportion in the sample were calculated.

7. Background contamination screening was carried out on the reference sequences obtained from the above screening. Firstly, if the reference sequence was not in the list of pathogenic species of clinical importance and accounted for less than 5% of all species in the sample, then it would be removed directly; Secondly, as to the species identified in samples with internal standard sequence detected, if they were also detected in the negative control sample, then calculate the probability that the ratio between the sequencing depth of these species and that of internal standard of the same clinical samples belongs to the same normal distribution of the ratios between these species and internal standard in all negative control samples. If the probability was greater than 0.05, the species was regarded as background contamination and removed from the test results or retained otherwise. The proportion of all species in the sample was then recalculated to obtain the final analysis results.

### 4. Experimental Results

This technical scheme was employed to test the original samples. The original test results included the nucleic acids of a known sequence (INQ internal standard) (relative abundance 1), Propionibacterium acnes (relative abundance 0.064) and Enterococcus faecalis (relative abundance 0.5781). By comparing the test results with the negative control samples of the same batch, based on the abundance relative to the nucleic acids of a known sequence, Propionibacterium acne could be determined as contamination, and the final result reported is Enterococcus faecalis, which was in agreement with the results obtained by microbiological culturing method.

| | | Identification | Sequencing | Relative | Proportion |
|---|---|---|---|---|---|
| Sample ID | Description of test results | result | depth (mean) | abundance | of species |
| INQ20M0053 | Original analysis results | Cutibacterium | 25.6868 | 0.064 | 0.039 |
| | (before contamination | acnes | | | |
| | was ruled out) | Enterococcus | 231.9836 | 0.578 | 0.352 |
| | | faecalis | | | |
| | | INQ internal | 401.2833 | 1 | 0.609 |
| | | standard | | | |
| INQ20MT005 | Negative control of same | Acinetobacter | 149.0041 | 0.358 | 0.118 |
| | batch | baumannii | | | |
| | | Acinetobacter | 242.0522 | 0.582 | 0.192 |
| | | calcoaccticus | | | |
| | | Burkholderia | 150.4143 | 0.362 | 0.120 |
| | | vietnamiensis | | | |
| | | Cutibacterium | 69.7435 | 0.168 | 0.055 |
| | | acnes | | | |
| | | INQ internal | 416.0538 | 1 | 0.331 |
| | | standard | | | |
| | | Sphingomonas | 81.512 | 0.196 | 0.065 |
| | | paucimobilis | | | |
| | | Sphingomonas | 101.3971 | 0.244 | 0.081 |
| | | trueperi | | | |
| | | Staphylococcus | 48.3199 | 0.116 | 0.038 |
| | | hominis | | | |
| INQ20M0053 | Final analysis results | Enterococcus | 231.9836 | 0.578 | 0.366 |
| | (after contamination was | faecalis | | | |
| | ruled out) | INQ internal | 401.2833 | 1 | 0.634 |
| | | standard | | | |

### Characteristic and Performance Data of Embodiments and Examples for Comparison

In the examples 3 to 8 of this invention, the average sequencing data obtained for each sample is 55,663 short sequencing reads, the valid data constitute more than 90% of the sequencing data in these embodiments, and the read coverage of the target sequence is close to 100%.

In example 7, the number of sequencing reads of the sample is 55,115, among which sequences of human origin accounted for 0.020% of all sequencing reads. A total of 7,707 short reads were mapped to the species Enterococcus faecium, accounting for 13.98% of all reads. The sequencing coverage for the target gene of the species was 99.9%, with an average sequencing depth of 349.04×. A total of 2,761 short reads were mapped to Escherichia coli, accounting for 5.01% of all reads. The sequencing coverage for the target gene sequence of the species was 99.8%, with an average sequencing depth of 140.85×.

In example 8, the number of sequencing reads of the sample is 67,797, among which sequences of human origin accounted for 9.50% of all sequencing reads. A total of 3,136 short reads were mapped to Escherichia coli, accounting for 4.63% of all reads. The sequencing coverage for the target gene of the species was 100%, with an average sequencing depth of 397.54×.

For comparison, metagenomic methods (mNGS) were used to test patient samples in a case report in the New England Journal of Medicine, 2014, 370 (25): 2408-2417. The sequencing data of the samples comprised 8,187,737 short reads, among which 98.7% were human genome sequences. A total of 475 short reads were mapped to the Leptospiraceae family, accounting for 0.0006% (0.6 per 10,000) of all data. The sequencing coverage rate of the genome of the species was 2.2%-3.7% with an average sequencing depth of 0.02-0.04×. The comparison shows that, the number of valid data, the proportion of valid data, the sequencing coverage and the sequencing depth of target genome sequences obtained by mNGS technology are far less than that of this technical scheme.

In example 9 of this invention, 89 clinical samples were tested. The positive rate of this technical scheme was 98.9% (88/89), which was higher than that of other non-NGS (bacterial culture combined with mass spectrometric identification) methods (59.6%, 53/89). Among samples tested positive by technologies other than this technical scheme, the agreement rate between the test results of this technical scheme and the former was 90.6% (48/53).

Meanwhile, in comparison with another case, Clinical Infectious Diseases (2018) 67 (S2): S231-40 reported that the positive rate of patient samples tested by metagenomic method was 38.2% (195/511). The positive rate of non-NGS method was 27.0% (138/511). Among samples tested positive by non-MNGS protocol, the agreement rate between the results of mNGS protocol and the former was 60.9% (84/138). Apparently, this technical scheme had demonstrated higher positive rate in clinical sample testing than the current technology. At the same time, compared to the related technology mNGS, this technical scheme has a higher agreement rate with traditional bacterial culturing method on samples tested positive.

The technical features of the above embodiments may be combined at will. To simplify the description, not all possible combinations of the technical features in the above embodiments have been described. However, as long as no contradiction exists in the combination of these technical features, they shall be considered within the scope of this description.

The above examples only illustrate several embodiments of this invention. It is to be understood that while the invention has been described in conjunction with the detailed description thereof, the foregoing description is intended to illustrate and not limit the scope of the invention, which is defined by the scope of the appended claims. It should be noted by anyone of ordinary skill in the art that, a number of modifications and improvements can be made without deviating from the concept of this invention, which are within the scope of protection of this invention. Other aspects, advantages, and modifications are within the scope of the following claims.

1. A method for identifying microbial species and obtaining related information by sequencing in a sample, comprising:
i) obtaining sequencing data by targeted enrichment of microbial characteristic nucleic acid sequences followed by next-generation sequencing of the enriched microbial characteristic nucleic acid sequences;
ii) comparing and analyzing said sequencing data with a characteristic sequence database containing reference sequences to identify microbial composition in said sample,
wherein said characteristic sequence database has been cluster processed in advance based on sequence similarity among reference sequences to obtain one-tier or multi-tier clusters, wherein there is at least one child seed in each cluster and there is one or more seeds as reference sequences in a bottom tier cluster;
wherein said comparing and analyzing includes:
a) removing low quality reads and reads containing sequencing adaptor sequences from said sequencing data;
b) performing sequence alignment between read sequences and seed sequences of said characteristic sequence database, removing completely duplicated reads generated by PCR amplification, performing statistical independence test between an event “has read aligned” and the seed sequences, selecting seed sequences associated with the event “has read aligned” as primary screened seed sequences;
c) performing sequence alignment between read sequences and said primary screened seed sequences, wherein said primary screened seed sequences do not compete with each other for read sequences, calculating reads coverage for each seed sequence, then evaluating coverage metrics of seed sequences to obtain secondary screened seed sequences;
d) performing sequence alignment between read sequences and said secondary screened seed sequences, wherein said secondary seed sequences compete against each other for read sequences, calculating read coverage metrics for each seed sequence, and removing iteratively seed sequences that do not meet a first threshold on read coverage metrics to obtain tertiary screened seed sequences;
e) merging said tertiary screened seed sequences and aligning them with read sequences, obtaining reference sequences that meet a second threshold by iterative screening, wherein the second threshold is more stringent than the first threshold of step d); and
f) based on said reference sequences and reads quantity obtained in step e), calculating content and proportions of reads at species level, wherein in the calculation, reads quantity of multiple reference sequences belonging to same species are added to obtain a total reads quantity for this species, and a species' relative proportion (abundance) is obtained by dividing the total reads quantity of each species by a sum of reads quantity of all species present in the sample.

2. The method of claim 1, wherein said microbial species include bacteria, archaea, fungi, mycoplasma, chlamydia, rickettsia, spirochete, and viruses, wherein characteristic nucleic acid sequences of RNA viruses are obtained by reverse transcription of viral RNA genomes to generate cDNA;
preferably, said samples to be tested are from microbial hosts or environmental samples containing microbial species:
said samples from microbial hosts include but are not limited to: at least one of feces, intestinal contents, skin, sputum, blood, saliva, dental plaque, urine, vaginal discharge, bile, bronchoalveolar lavage fluid, cerebrospinal fluid, pleural fluid, ascites, pelvic effusion, pus, and rumen; and
said environmental samples containing microbial species include but are not limited to: at least one of internal and external surfaces of objects, domestic water, medical water, industrial water, food, beverage, fertilizer, waste water, volcanic ash, frozen soil, silt, soil, compost, polluted river, aquaculture water bodies and air.

3. The method of claim 1, wherein the targeted enrichment in step i) is by a method including PCR, nucleic acid probe hybridization capture, biotin labeling capture, digoxin labeling capture, isotope labeling capture, magnetic bead capture, antibody capture, CRISPR/Cas technologies, or a combination thereof, wherein reaction mode can be in a liquid, on a solid surface, or a combination thereof.

4. The method of claim 1, wherein said sample is from microbial hosts, wherein step a) further includes: removing nucleic acid sequencing data of said hosts in said sample.

5. The method of claim 4, wherein said hosts are human beings.

6. The method of claim 1, wherein step d), after said iterative removal of seed sequences whose read coverage metrics do not meet the first threshold, further includes screening reference sequences within the cluster:
performing sequence alignment between reads and reference sequences of the cluster obtained in last step to which each seed belongs, wherein reference sequences within the same cluster compete for reads;
calculating read coverage for each reference sequence and filtering the reference sequence according to a read coverage metrics; and
removing iteratively seed sequences of poor read coverage using a threshold, wherein the threshold is more stringent than that in steps before step d).

7. The method of claim 1, wherein step f) is followed by step g): removing nucleic acid sequencing data of background contaminating species in experimental environment.

8. The method of claim 1, wherein in step b), said statistical independence test is Fischer's exact test, which includes:
labelling reference sequences aligned with reads of more than a certain number as “has read aligned” or as “no read aligned” otherwise;
according to clustering hierarchical relationship of seeds in the reference sequence database, performing statistical testing for each seed in the clustering tree to determine whether it is significantly enriched with reference sequences labeled as “has read aligned” in its leaf nodes, and
identifying seeds meeting requirements via screening tier by tier.

9. The method of claim 1, wherein said characteristic sequence database is constructed by:
obtaining public databases containing reference sequences of said characteristic sequences, and removing sequences at both ends of the amplification primers of said reference sequences in said database to obtain a first database;
based on intra-species sequence similarity, performing base correction on said reference sequences with ambiguous bases in said first database, and removing redundant reference sequences of 100% sequence similarity based on their species annotations and sequence similarity to obtain a second database; and
performing clustering on said reference sequences in said second database based on sequence similarity.

10. The method of claim 9, wherein while constructing said first database, removing sequences of said amplification primers and sequences at both ends of and external to said amplification primers of said reference sequences in said database.

11. The method of claim 9, wherein while constructing said second database, it also includes:
performing Blast search for each reference sequence against NCBI NT/NR database, a set of matched reference sequences is screened from the NCBI NT/NR database according to rules based on sequence similarity and/or coverage; and
selecting most representative species classification from species annotation of said set of matched reference sequences, and use classification information to correct species annotation of the reference sequence.

12. The method of claim 9, wherein said clustering includes a first clustering:
performing clustering on all non-redundant reference sequences based on sequence similarity; or
1) performing clustering based on sequence similarity of non-redundant reference sequences within same species, and
2) perform clustering on seeds obtained in 1) based on sequence similarity, merging the seeds that belong to different species but are clustered into the same cluster with their child sequences, then performing clustering according to 99.5% sequence similarity, and replacing old clusters involving in re-clustering computation with newly formed clusters.

13. The method of claim 12, wherein said clustering also includes a second clustering:
in case that there are too many child sequences in a cluster, splitting the cluster obtained by said first clustering using a sequence similarity standard higher than that used in said first clustering, and replacing clusters before splitting with new clusters formed after splitting.

14. The method of claim 13, wherein said clustering also includes a third clustering:
according to different sequence similarity thresholds, performing hierarchical clustering on seed reference sequences of the clusters obtained by said second clustering to construct a hierarchical tree.

15. The method of claim 1, wherein said microbial characteristic nucleic acid sequences include sequences of 16S rRNA gene, 18S rRNA gene, ITS nucleic acid sequence, RNA dependent RNA polymerase (RdRp) gene of RNA viruses, viral capsid protein coding gene, and pol gene of retrovirus, or other full-length sequences of one or more among nucleic acid sequences capable of reflecting microbial taxonomic characteristics.

16. A device for identifying microbial species and obtaining related information by sequencing in a sample, said device comprises:
a sequencing data acquisition module, which is used to obtain sequencing data, wherein said sequencing data is obtained by targeted enrichment of microbial characteristic nucleic acid sequences followed by next-generation sequencing of the enriched microbial characteristic nucleic acid sequences;
a characteristic sequence database construction module, which is used to carry out clustering on said characteristic sequences to obtain characteristic sequence database, wherein said characteristic sequence database contains one or more tiers of clusters, wherein there is at least one child seed in each tier cluster and there is one or several seeds as reference sequences in a bottom tier cluster; and
a comparative analysis module, which is used to carry out sequence alignment between said sequencing data and said characteristic sequence database to identify a microbial composition in said sample, said comparative analysis module includes:
a) a first module, which is used to remove low-quality reads and those containing sequencing adaptor sequences from the sequencing data;
b) a second module, which is used to perform sequence alignment between read sequences and seed sequences of said characteristic sequence database, remove completely duplicated reads generated by PCR amplification, and perform statistical independence test between an event “has read aligned” and the seeds sequence, and select seed sequences associated with the event “has read aligned” as primary screened seed sequences;
c) a third module, which is used to perform sequence alignment between read sequences and said primary screened seed sequences, wherein said primary screened seed sequences do not compete for reads, calculate read coverage for each seed sequence, then calculate and evaluate coverage metrics of seed sequences to obtain secondary screened seed sequences;
d) a fourth module, which is used to perform sequence alignment between read sequences and the secondary screened seed sequences, wherein seed sequences compete among each other for reads, calculate read coverage metrics for each seed sequence, and remove iteratively seed sequences that do not meet a threshold on read coverage metrics to obtain tertiary screened seed sequences;
e) a fifth module, which is used to merge said tertiary screened seed sequences and align them with reads, obtain reference sequences that meet a second threshold by iterative screening, wherein the second threshold is more stringent than the first threshold of step d); and
f) a sixth module, which is used to, based on said reference sequences and their read quantity obtained in step e), calculate content and proportions of reads at species level, wherein in the calculation, read quantity of multiple reference sequences belonging to same species to obtain a total read quantity for this species, and a species' relative proportion (abundance) is obtained by dividing total read quantity of each species by a sum of read quantity of all species present in the sample.

17. A computer readable storage medium, wherein said computer readable storage medium is used to store computer instructions, programs, code sets or instruction sets which, when executed on a computer, causes the computer to perform methods of claim 1.

18. An electronic device comprising:
one or more processors; and
a storage device that stores one or more programs,
wherein said one or more programs is executed by said one or more processors to implement said method of claim 1.

19. (canceled)


SEQUENCE LISTING (17 sequences)

SEQ ID NO: 1 - DNA, 20 bp, Artificial Sequence
  This nucleotide sequence was designed and synthesized as a primer for use in polymerase chain reaction (PCR) amplification. It does not occur in nature.
agagtttgatcmtggctcag

SEQ ID NO: 2 - DNA, 20 bp, Artificial Sequence
  This nucleotide sequence was designed and synthesized as a primer for use in polymerase chain reaction (PCR) amplification. It does not occur in nature.
agagtttgatcctggctcag

SEQ ID NO: 3 - DNA, 18 bp, Artificial Sequence
  This nucleotide sequence was designed and synthesized as a primer for use in polymerase chain reaction (PCR) amplification. It does not occur in nature.
ctcctacgggaggcagca

SEQ ID NO: 4 - DNA, 16 bp, Artificial Sequence
  This nucleotide sequence was designed and synthesized as a primer for use in polymerase chain reaction (PCR) amplification. It does not occur in nature.
gtgccagcmgccgcgg

SEQ ID NO: 5 - DNA, 20 bp, Artificial Sequence
  This nucleotide sequence was designed and synthesized as a primer for use in polymerase chain reaction (PCR) amplification. It does not occur in nature.
aaactyaaakgaattgacgg

SEQ ID NO: 6 - DNA, 16 bp, Artificial Sequence
  This nucleotide sequence was designed and synthesized as a primer for use in polymerase chain reaction (PCR) amplification. It does not occur in nature.
gcaacgagcgcaaccc

SEQ ID NO: 7 - DNA, 20 bp, Artificial Sequence
  This nucleotide sequence was designed and synthesized as a primer for use in polymerase chain reaction (PCR) amplification. It does not occur in nature.
agagtttgatcatggctcag

SEQ ID NO: 8 - DNA, 25 bp, Artificial Sequence
  This nucleotide sequence was designed and synthesized as a primer for use in polymerase chain reaction (PCR) amplification. It does not occur in nature.
taactgaagagtttgatcctggctc

SEQ ID NO: 9 - DNA, 19 bp, Artificial Sequence
  This nucleotide sequence was designed and synthesized as a primer for use in polymerase chain reaction (PCR) amplification. It does not occur in nature.
ggttaccttgttacgactt

SEQ ID NO: 10 - DNA, 21 bp, Artificial Sequence
  This nucleotide sequence was designed and synthesized as a primer for use in polymerase chain reaction (PCR) amplification. It does not occur in nature.
ggytaccttgttacgacactt

SEQ ID NO: 11 - DNA, 16 bp, Artificial Sequence
  This nucleotide sequence was designed and synthesized as a primer for use in polymerase chain reaction (PCR) amplification. It does not occur in nature.
ctgctgcsycccgtag

SEQ ID NO: 12 - DNA, 21 bp, Artificial Sequence
  This nucleotide sequence was designed and synthesized as a primer for use in polymerase chain reaction (PCR) amplification. It does not occur in nature.
gwattaaccgcggckgctgtg

SEQ ID NO: 13 - DNA, 20 bp, Artificial Sequence
  This nucleotide sequence was designed and synthesized as a primer for use in polymerase chain reaction (PCR) amplification. It does not occur in nature.
ccgtcaattcmtttragttt

SEQ ID NO: 14 - DNA, 15 bp, Artificial Sequence
  This nucleotide sequence was designed and synthesized as a primer for use in polymerase chain reaction (PCR) amplification. It does not occur in nature.
gggttgcgctcgttg

SEQ ID NO: 15 - DNA, 17 bp, Artificial Sequence
  This nucleotide sequence was designed and synthesized as a primer for use in polymerase chain reaction (PCR) amplification. It does not occur in nature.
aaggaggtgwtccarcc

SEQ ID NO: 16 - DNA, 24 bp, Artificial Sequence
  This nucleotide sequence was designed and synthesized as a primer for use in polymerase chain reaction (PCR) amplification. It does not occur in nature.
tagggttaccttgttacgacactt

SEQ ID NO: 17 - DNA, 24 bp, Artificial Sequence
  This nucleotide sequence was designed and synthesized as a primer for use in polymerase chain reaction (PCR) amplification. It does not occur in nature.
tacggttaccttgttacgacactt